SEQUENCE LISTING (20 sequences)

SEQ ID NO: 1 - DNA, 2945 bp, Cricetulus griseus
caaggctgaggcagcgccatctggtactccctgcccccctttctctcttgataactgagattataggttggctccacagctgccttggctggaatttccatctgataaaatgaagtctggagccactaggattgcaccaggggcttagtaaaagctcccccacccccaccccaatgaatggggcttctagtgagattcctggaaagggagggcactaagcagttggtaatttagctggaaatcttgtttttcctcaggcacaaggcttcatctggattcctaggtgaggagggcttcttcgctgttctcccctctctcccacactcagaatttcctttcttcctatctggccctgcttttctttcttattttgagacggggtttcactaagttgtcaggctagcctttactcacttactctggagtagctagggtcgaaagcactctccacccacccgatctcccagaaagcacacttctcaaacagactccggctcttcatttttaaacttaaatttacaaattggttgacagtctcatattccaacctggtctagcagaactttaggtcctccacttcctgcgtgccgactataaagtgtgagccaccactcccagttacacagtagcttagaaaaagaggacaggattgagtccaagataaacgttgtcatgtctttgtgccttacgtttctaatataggggtgagtattagaaatacggttaagaagtatgcactttgaatgtaaagtgctttttacatttacataaaaagcattgtattaaaatgatcttcttggacagtaacaaaaagtacttccctccaggaactccaagccaacattcttcccaagatacaaatacagaagaaaaaaaaatattcccttattagtgtctcagtaattactggctatgatgaggacaaagcctccccagtcttctgatagcctttctcgcgtggtatggagaatttccaatcaaacaccgtaagtgaaagttgctacggtattcttttaaaaaacaacctaactaatagctgtcctgcccttttgaaaggacacgtggaaagtgctggagagacgggcttaatgacaaggccccaaacgaacgccctgcctccatgccttgcagtttcatagtccaagcactgtcacgttttcaaggactctgcgcggtcgagcgtttaatttttggtataagtcaagtcaggggagacattacctcatgttactgaaaataaaaggtcgagatcgccgcatcaaggacacagggctgccggaaacgaggattgccaaggcctagctcactctcagggggaaaaaaatatacccttttgcacattttttttcttttttcacaaagtgagaggtctggtggggcagggaggggacggaggccacgctatcctagcccgggggtggggaggggcgctcggctctggggtcggccaagaagacgaggccatttctttcactgagggtcacaaccgagcatcctggggacctgtacaagtcaggcacgtctaacctccgcactggggggctgcggactgacgtgttccatttctcagagacaccgcgcggcggccgagccatctccaaacgcggcgccagacccagctatcctcgcggtccccactagtctcagactcagacaccgggggcgcgcgaagcctcaggccgctggcccggcagcccgacaggctgagggagaaggacccaggacggggtgggggaggggcgcaacaaccttcgcggccattttgtcctcgccccacttccgtcttctcttcccggactccccctccctcaggctcttctctccggcggtccggggtcccgagaggccccgaacgcggagaacactgctctcattggtccgctgtgaatccacccgcctcccttcgtcggcttccttccgctccctcccgcacgcgctgactgggcgcgcatgcgtacaggctgccgagtccctcccctcccccgccagccgtgagtgacagaggactcccgcgcgcgggctctcggcgccccgccctcccctttcccccgggccgttcggagagcgggggcgagcatgaaagttccagaacgctgcggtcgtgcgtcatcgccaggtggggcggggcgagggcgggacctaggagagcgtctcgggcgttcattggtgctgttcgaaaagccccatcgtccattggctaaggagccatgataggcagcgggacggggcgggatccttctggaaggttctgagacagggtataagagacagtgaggcgggcggaaaccggtctcattgaacgcggaggcagcttgtgaggttttgtgtggtctcgtcgccagcacagccgggcctacaggcaaggtgaggcctggggcttgggtggtggcgggtggcctgctttactgcagagcggcggggcactgaggcggcgggcgggggcttaggccgcggcctccggggacccgggctgcagctgaggaggatgggcttaggtagggcggcgccgccatggccttgaggctcgtcgcggcgggggaggggtgccgcagccgcccgcgccgctccctttcacccgtctgcggattaaacgagagggtttctgaccgcggcccaagggcgagctgcttggaatatttcagcaggcgagtctggcgccgcaatgcgcggtcctggcggctggaaggggggaggggttgggttctccgagttgaggccacgtgctcaaccattgtcgcagaccgtcaggacagatggttcttcagccaggggcgggctctgccagggttggcacggctaccggactgcagcagtgccgccctgactgagccttttgcgtggcctcaggtgataggtctgggctgatgaggacatacggattgcgggtctggaagctggggaatagctgcttagccgttttcgtgtttggttttctttcagcaacc

SEQ ID NO: 2 - DNA, 2000 bp, Homo sapiens
atacaaacgttcagaaagtctaaatcaaggacgcacggctactggaacctatgattcctaatgcctggttcacctaaccaagaacacactcgccaccatttaaacctgtttattttataaacaacggggaaatgtagggcttgggctagttggggaaggactgagccaagaagccgaatctgttctagacggaggtgggaaggggcctaacggtcagggctcagagaagcggtttcgccttcttttccgcggagggatccagacacgaccgcgtcctgaggcggttctgaaaagccgggccccttaaacctctgcgtcgggggctgcctcccgacagttgccgtagggaaatgcaggtgctgggaggcctgccgagctaacccgccccaccccgcggcggcctggcggctccctccaatcccaatcctggggggccgtgagcgagcagccctagtggcaccctgaagccggaaccttcgccccagcccctcggggtcagcccttgtccaaagccacgctatgaaatacagattcgcgaaacccggggcacttagaggcccgtctggaacacgaaggtggggcatggaaaggaggattcggacgtgggtgggggaggggagcaacaaccttcgcagccattttgtcctcgctccacttccgtcttctcctgcccggctcccgcgcccaaacccctcccttcaggccccgcgcgattccgcccccagttctgtgccggccaagatcccggctagcgccgctatcattggttagttccaagtttgcccgcccctcttcctcctcctttttccgccccctccctcccgcggaagctgggggcgcatgcgtagaggtggacgctcccctcccccgcccggggtaactgaggactcccgcgcgcggactcgctgcgccccaccctccctttccccggggccgtccggagagcgggggcgagcttgaaagttccagaacgctgcggtgagtgcgttatcgtgaggcggagcgcggtggggtgggtgcggaagggggcgaggcccgaggagtggagccgggcttgtgattgggtcttgtaagggcagccgggcgtctattggccggggaagccgtaatggcaggcagcaggggcgggccccttctggaaggttctaagatagggtataagaggcagggtggcgggcggaaaccggtctcattgaactcgcctgcagctcttgggttttttgtggcttccttcgttattggagccaggcctacaccccaggtaaaacctctgctcaagagttgggttgtgggtctgggagcgtgcagcctccacacaggcctgttgggcttgctgaggcttgggggttctgagaatctcgtcgaggcgagtgtgcggctccttctaccggcttaaagggcctcagttttcggtgggatggcagcggtatttggttgcagccggcaggacggaaatgtagggagtgggccgcagtggccccaggggaggctgggagacgcccggcggccgcgtggcgggggagggttgctgcatcggtttgcctggcgcgcggggaagtggagccagcgttttctttcacccagttccctgcttagtccagtcccaccgtggttcttcagagctgttcttggcgtgcttccagtatgggggtacattccggagtagttaaaagcccgttgactcccgggggcactggcacctggcgagggaggggaacagacagtgctcagttcggggtaagaccacgtgttgagcaacgccccacgccgtctgggtagatgggtccttcatctagggcgtgctctgctgcggttggcacggcaacctggactgcagcactagttctggacctcgcgcgtgcttagacaggaggtgatgggcactattacctcttggcagtggccatacgtttttcctggttaagtgttctgttaagggatgagggaaatattttgattaattgaatttttaaaccagatttttctttttttcagcaacc

SEQ ID NO: 3 - DNA, 2000 bp, Mus musculus
agaccttccaatttaaacgccacaacagtattctttaaaaaataaaaagacaacctatctaactaataaagagctgcactccctgcctatttgaaaggacgcgtggaaagtgctagagagccgggtgtgacgaggcccggaacgcccagcctccaggcactgcggtttcagagtctaagcactcgctccggccattggctcagtcacgttttcaaggactcagcgcggccgggcgtttaatttttggtaaaagtcaagtcaggagagacattacctcatgttactgaaaataaaaaggtcgcgatcactctatcaaggacacggggctgccggaaaccaggaatgccagtgcctagctcacccacaaagggacaaaaaacatttatctttcgtacatttattccattaactgatgagtctggtggagcagagaggggacagaggtcacgctgtcccctgtgggacagcggaggtggggaggggcgttcggctcaggggtaagcagagaggactgggccatttctttgatagaggatcacaaacgatcctggggacatgtggcgagccaggcagatcgagcctccgcactggggggggggggaatcgaggtgtcccattttgtggggggtgaggggtggggaaagccagcgagatagcgcggcggcttagcctcccacaagctggcagcacgaaacaagaagcctagcagtgctccttagtctcaggctggggtggaccaagcccgaggccgggaaaccgccgggctcagggagaaggaccgagggtcgtgtgggggaggggcgcaacaaccttcgcagccattttgtcctcgccccacttccgtcttctcttcccggactccccctccctcaggctcttctctcaggcagcccggggtcccaagaggtagggatccggccgaacgctgctctcattggtccgtcgcggaggtacccgccttcctcccagggcatccttccgccccctcccgcctgcgctgccgggtcgcgcacgcgcactcgccggcgaccctctcccctcccccagcagctctgagtgacagaggactcccgcgcgcgggctcccggcgccccgccttctccgctccccccgggccgttcccagagcgggggcgagcgtgaaagttccagaacgccgcggtcgtgcgtcatcgcgaggcggggcggggagaaggcgggacctaggcgagcgttctggctcttcattagtgcagtcggcaagggctggtcgtccattggctaagaagctatgatcggcagcgggatggggcggcacccttctggaaggttctaagacagggtataagagagagggaagcgggcggaaaccggtctcattgaacgcggaggcagctgcctggcatttgtgtggtctcgtcgtcagcgcagctgggcctacacacaaggtgaggcatgggactcgggggtttgcgggggtccggggtggtgcaggagtcccggggagtctcttaggcggcggctgcagctcaggccgtcaccaccagggacagagactgcagcagatccggaggatgggctgagtcagggcggcgccgccatggccttgtggctccgcgtggcgggggaggggagccacaggcgccctggtctttttatcctgtctgtgacttcaaatataaggcttctgcccggcgcccacagggcagggccccccgaggtgggaaagggggatcctggctcccgcggtgcgcggtccgggtggggggggttcggggggcgctcggaggtggccacgtgctccaccattgtcgcagacctttaggacagatggttccttgagccttgggcgggctgtgccaggcttggcacagaccacctatactgcagcactgctgccacgagcctgccgcgtggcttcaggtgataggtctgggtcaccaagtccagacccactgcggactggataaaagccggaaagcgagtgcttagcccccctcctttttttttctaatagcaacc

SEQ ID NO: 4 - DNA, 2000 bp, Rattus norvegicus
cttttgatagccttcctcacatggtctggagaccttccaattcaaacgctacaactaaaagttgttgcagtatatatatttaaaaaaaaagaaaaaaaccaacctatctaactaataaatatctgttctccctgcccatttgaaaggacacgtggaaagtgctagagacaggcttggtgtgacaaggcccggaacgccctgcctccatgcactgcggtttcctagtccgagcatcggctctggccactggctcagtcacgttttcaaggactctgcgcagtcgggcgtttaatttttggtaaaagtcaagtcaggagagacattacctcatgttgctgaaaataaaaggtcgagatcactcaatcaaggacacagggctgccggaaaccaggattgccaatgcctagctcacccacaaggggaaataaattatccttttgaacatttattccataaagtgataagtccagtggggcagagagggaatagaggccacgctgtcctcgtccagagatggggaggggcgctcggctccgggggtcagcaaagaggactggaccatttctttcacagaggattaccaacgaggaccctggggacatgtgacaagtcaggcattatcgagcccccacactgggggatcgacatgttccattttgctggggggacagcaagggaggtcgctcggcggcctagccttccacaagagaggctccacgaaacaagcctagcagtgctccttagtctccggctcggaagccgtgggtggacgaaaccggaggccgggaaactgctggactcacggagaaggaccgaggggtggggtgggggaggggtgcaacaaccttcgcagccattttgtcctcgccccacttccgtcttctcttcccggactccccctccctcaggctcttctttcaggctgcacagggtcccaagaggaacagatagcgccgagcactgctgccattggtccgttgcgggtgtacccgccctccttcatgggcatccttccgctccctcccgcctgcgctgacgggtcacgcatgcgtactcggtgccgacgctctcccttcccccaccagctctgagtgacagaggactcccgcgcgcgggctctcggcgccccgccctcccctttcccccgggccgttccgagagcgggggcgagcgtgaaagttccagaacgctgcggtcgtgcgtcatcgcccggaggggcggagtggaggcgggtcctaggagagcgttctggttgttcattagtgcactccggagaacctggccgtccattggctaaggagctctgatcggcagcgggatggggcggaacccttctggaaggttctaagacagggtataagagacagtgaagcgggcggaaaccggtctcattgaacgcggagccagctctcgggggtctctgtgtggtctcgtcatcagcacagctgggcctacacgcaaggtgaggcatgggacttgggtgttggtgcgtggccggtgtggtgcaggggtcagtgggatgcttaggcggcgggtgcggcttaggccgctgttgccagagacggaggctgctgctgaaggggaggatgagctgagtcagggcggcgccgccatggccttaagcctccgcatggcgggggaggggagctcaggattccggggccttttcttttttctgtctgcaagctaaaggagggtctcttccgcgcgttcccagggcacgggtttcagagatggaagaggcgatccagcctcccgcagtgcgcggtcctgggggtggggggggccctcggaggtggccacgtgctcagccattgtcgcagaccttttggacagatggttccttcagcctagggcgggctgtgccaagcctggcgcaggccactcaaactgcagcagtgtcgcctgcgtggcttcaggtgattgataggtctgggccggtgcagagtgaaagcagggagatgaccgcttagtcctttttttttttccttctagcaacc

SEQ ID NO: 5 - DNA, 1926 bp, Cricetulus griseus
aacaacctaactaatagctgtcctgcccttttgaaaggacacgtggaaagtgctggagagacgggcttaatgacaaggccccaaacgaacgccctgcctccatgccttgcagtttcatagtccaagcactgtcacgttttcaaggactctgcgcggtcgagcgtttaatttttggtataagtcaagtcaggggagacattacctcatgttactgaaaataaaaggtcgagatcgccgcatcaaggacacagggctgccggaaacgaggattgccaaggcctagctcactctcagggggaaaaaaatatacccttttgcacattttttttcttttttcacaaagtgagaggtctggtggggcagggaggggacggaggccacgctatcctagcccgggggtggggaggggcgctcggctctggggtcggccaagaagacgaggccatttctttcactgagggtcacaaccgagcatcctggggacctgtacaagtcaggcacgtctaacctccgcactggggggctgcggactgacgtgttccatttctcagagacaccgcgcggcggccgagccatctccaaacgcggcgccagacccagctatcctcgcggtccccactagtctcagactcagacaccgggggcgcgcgaagcctcaggccgctggcccggcagcccgacaggctgagggagaaggacccaggacggggtgggggaggggcgcaacaaccttcgcggccattttgtcctcgccccacttccgtcttctcttcccggactccccctccctcaggctcttctctccggcggtccggggtcccgagaggccccgaacgcggagaacactgctctcattggtccgctgtgaatccacccgcctcccttcgtcggcttccttccgctccctcccgcacgcgctgactgggcgcgcatgcgtacaggctgccgagtccctcccctcccccgccagccgtgagtgacagaggactcccgcgcgcgggctctcggcgccccgccctcccctttcccccgggccgttcggagagcgggggcgagcatgaaagttccagaacgctgcggtcgtgcgtcatcgccaggtggggcggggcgagggcgggacctaggagagcgtctcgggcgttcattggtgctgttcgaaaagccccatcgtccattggctaaggagccatgataggcagcgggacggggcgggatccttctggaaggttctgagacagggtataagagacagtgaggcgggcggaaaccggtctcattgaacgcggaggcagcttgtgaggttttgtgtggtctcgtcgccagcacagccgggcctacaggcaaggtgaggcctggggcttgggtggtggcgggtggcctgctttactgcagagcggcggggcactgaggcggcgggcgggggcttaggccgcggcctccggggacccgggctgcagctgaggaggatgggcttaggtagggcggcgccgccatggccttgaggctcgtcgcggcgggggaggggtgccgcagccgcccgcgccgctccctttcacccgtctgcggattaaacgagagggtttctgaccgcggcccaagggcgagctgcttggaatatttcagcaggcgagtctggcgccgcaatgcgcggtcctggcggctggaaggggggaggggttgggttctccgagttgaggccacgtgctcaaccattgtcgcagaccgtcaggacagatggttcttcagccaggggcgggctctgccagggttggcacggctaccggactgcagcagtgccgccctgactgagccttttgcgtggcctcaggtgataggtctgggctgatgaggacatacggattgcgggtctggaagctggggaatagctgcttagccgttttcgtgtttggttttctttcagcaacc

SEQ ID NO: 6 - DNA, 1199 bp, Cricetulus griseus
aaccttcgcggccattttgtcctcgccccacttccgtcttctcttcccggactccccctccctcaggctcttctctccggcggtccggggtcccgagaggccccgaacgcggagaacactgctctcattggtccgctgtgaatccacccgcctcccttcgtcggcttccttccgctccctcccgcacgcgctgactgggcgcgcatgcgtacaggctgccgagtccctcccctcccccgccagccgtgagtgacagaggactcccgcgcgcgggctctcggcgccccgccctcccctttcccccgggccgttcggagagcgggggcgagcatgaaagttccagaacgctgcggtcgtgcgtcatcgccaggtggggcggggcgagggcgggacctaggagagcgtctcgggcgttcattggtgctgttcgaaaagccccatcgtccattggctaaggagccatgataggcagcgggacggggcgggatccttctggaaggttctgagacagggtataagagacagtgaggcgggcggaaaccggtctcattgaacgcggaggcagcttgtgaggttttgtgtggtctcgtcgccagcacagccgggcctacaggcaaggtgaggcctggggcttgggtggtggcgggtggcctgctttactgcagagcggcggggcactgaggcggcgggcgggggcttaggccgcggcctccggggacccgggctgcagctgaggaggatgggcttaggtagggcggcgccgccatggccttgaggctcgtcgcggcgggggaggggtgccgcagccgcccgcgccgctccctttcacccgtctgcggattaaacgagagggtttctgaccgcggcccaagggcgagctgcttggaatatttcagcaggcgagtctggcgccgcaatgcgcggtcctggcggctggaaggggggaggggttgggttctccgagttgaggccacgtgctcaaccattgtcgcagaccgtcaggacagatggttcttcagccaggggcgggctctgccagggttggcacggctaccggactgcagcagtgccgccctgactgagccttttgcgtggcctcaggtgataggtctgggctgatgaggacatacggattgcgggtctggaagctggggaatagctgcttagccgttttcgtgtttggttttctttcagcaacc

SEQ ID NO: 7 - DNA, 8450 bp, Homo sapiens
attttgcttgaaaggatagcatcaaggaagtgaaatgacaacccacagaatgagagataatttttgcaaatcatgtatctgataagggacctgtagtcagaatatgcaaagaacccttacaattcaataagacaacccaatttaaaaacaggcaaaggatgtgaataggcatttctccaaagatacggaaaaacggccaataagcacataaaaagatgctcaaaatcatttgccatttgggaaatgcaatcaaaaccacaatgaggtatcacttcacgcccattagggtggctatagatcagaaagtcagataacatgtgttggcaagcacatggaaacactgaagtccttacacactgctggtaggaatgtaaaatggtgcagccactgtggaaaacagttttccaatttctcaaaatgttaaacacagttatcatacacccaagcaattctactcttaggtatatacccaagagaaatgaaaacatatgtcttcaccagaacttgctgttcacagcagcattatgcataatagaccaaaagtggaaacaactcaactgcccatcaactggtgaatggataagtaaaatgtgatgtaaccagtcattggactgtcattcattaataaaaagaacaaggtactgattcatgttctaacatgagtgaatcttgaaaacactatgctaaattaaagaagccagtcacaaaaggccgtgtattgcatgattttatatatacatgaacttttatatatatataattatatatattatatataattttatatatataaatttctatatataaatatataaaatcatatatatgatatatattttttcatatacatcatatatatttacaaaaattatatatcatatatcatatgatatatgagatatatatcatgatatatatgatatatgatatatatcatatgagatatatgatatcatgagatatatgatatcatatgatatatatgatatagatatcatatgatatatatataatatatatatgatagatatattatatatgatagatatgatagatatcatattatatatgatagatatgatagatatcatattatatatgatagatatagatatcatattatatatgatagatatgatagatatcatattatatatgatagatatgatagatatcatattatatatgatagatatgatagatatcatattatatatgatagatatgatagatatcatattatatatgatagatatgatagatatcatattatatatgatagatatgatagatatcatattatatatgatagatatgatagatatcatattatatatgatagatatgatagatatcatattatatatgatagatatgatagatatcatattatatatgatagatatgatagatatcatattatatatgatagatatgatagatatcatattatatatgatatcatatatataccacatacatcatatatacatcatatatacatcatatatatcatacatatatatgaactttccagaataggtatatcaataaagacaggaagtatacaagtggttgccacagcctgagaggagcagggaatggtgagtgactgctaatggatatggcactttttttggggggtgatgaaaatgttctggtcagacaatggcaattacaaaactgtatacacacgaaaaaccaaagaatcacacactttaaaagggaggatttagctcggcatggtggcatgcgcctgtactcccagttactcgggaggctgaagcaggactgcttagagcccaggacttcaaggctgcagcgagctatgatcgctccactgcactccaacaaggatgacagtgcgagacccgttttctaaataataataataataataataataaataacccaaggtacccagttcacatgcaaaaccactggtaaacataaattatctccaagtaatctagaaagaaaatgagcacataagacgtcttctaaaaacacacatatatttctttacatgttacatttaacgtaaaaatcagctatgcagaagttacatgaacattttatgttggaaaggtaaatgactattattaatacagaatggttaagtacatttatgtttttatgtacaaacgcataaaaggaaaagcatccttaaaataaacaccatcaatggctcctcggtggtcacaaaacaaaatcctcacacctttgtcttccttcacaattgagctttatccaccttttcaggcttatctcccattattacctgacacaaacttgggtgggccagagtttccactgaccatcccccgactattcatccaacactatgttcactgcctcccattcctgaccatttgccttttgtcttcaactaattctggggacgttttgtccaaataaatgatccatattcttgaaggctggaatcaagtcctattacaaatatattttctcaccctctccagagcatagcaacccagcatctactggcctctcacagctctaaccatccacaaccctaagctggcttctcatcaaacgggtacttttcaccacccaaattcaattaattcactcttacaataatgaagaatagtcgcctacagcctaccttttccagccttgattcaatcatttatcaattttatcttcaaagtccctcacttcagggagatgatatatcagctttcacccagagtcctaaagaaaacagcactcttgccaatgacatagtgccacctagtggcaacataaggtaaatcacagtggcagtagaaggatctccacactacttttacaggaatgcactgcaggtaaaaaataagaagctacagtactgtttggcaggacaatttgtttcatacgtgcatactatcgccctgactaaattaactcgcaagtcttacaggtattatttgttttcagttccatgcacagattagccatttagtacttactaaatcaaactcaatttctgaagtgtcttacaccaatatattcatgcacatatggttaaaattttccttgaggatctatcatgtgagagtgtggcttattataacaagtaaacagaacaaataaatacaaaatgaaaagaaatcgtatgatttactcgcatataagggagcttgttgtggattaagtttcatgacccaggacactgaaacagaaatggaataaatgagaataaaattaaaagttgtcatcaaaaatatagaagccatctaaagacctaggtgtcaagcatagctctatgagtacaatcccgtgcctgagattaccatatgcccagctgtatgctatacactaagagatttaggaaggaagcggggtcagggattgaccccagactccatcttttcaagtggggaagaaagatcttccgattgaaaaataaaggcaaaaaaggcttcaccgtcacagaagtttcaacaaccaacaggatatttaaaacagttatcaaagcaaaaccattgtatgttcacttacatttttacatagtccctcaaactcacaaaatgctgtttactcagggacttcttccggtcttactagggagcctggaaagtgacgggaggattgcaagggaccactagaaccctcttcctcaattccccttctctgagaagggaggctacagcttgcctctctaaccactaaaaggcatgaccctcctcaaagttaatagccggattccctgatagatattttcactaaatgaattctcataaaactctcactaagatttagagaaggcttccagggttgaattcctgaacattaagaacagcatgttttttaaaagtttaacttggtgattggaccaggacttcatctaggctatgaatgctcagaatggtaggtcctttaccaaacagcttgagtttgtgtataaagtgatctcatcctcttaagagtcagagaaacagaaccaagcgacttcactataatttgatctgaggaagtttcttactcacaataggtaaatgaaggcacatactaaccagcaatataaacaacaatatcaagtgtcattcacacatgcaaaaaacagacaaaatcccaaactctgtgttctaacaaatcgcaaaaacctcactaacaataaattgaaatgaccaaatgtttggactgaaaagcaatgccttggtagcctagccatgcctaactcaaataacagaaccatctcgatgttaaaatcctcacagatcaagctgtgtatgtctcgggtcaagacttcgccaaaaagcagtgagcacacacttaagagggaaaaaatctacctcagcctcctaaatgcaatcatctctacacgagttgcaggccccaagcttcaacgtgttctgctggacaacgcagtagaaagctgacaagcaggtggccttcccacactgactgaaccacctccatgcccatgtccattcattttcttgcccaccccatgtgctataacagacctcctggctcagggcactctttccttcctgactgccttcacttaatgactttgtacttttaggtgcaaaaattatctgcagaaatccacactgaaaaccaagcttgagaaaggcagcaataaccaacatttttacaagaagaacaaggtcaatatcaagcccatcagattcaaatagcaagcatggatgaaaatgaaagattgaaaggcttgagtgccttcttaatgtattaaatatccatttaatttacaattaagctcactgtgctcactggccttttaatcagctttccaggtcctgctcagacttgcctaggacatgggaatgaaagaacctatacatttatggaccaatctaccttaactaacttgtcaagtgttcctgcatcaagcagaagaaacatcagtgaaactgatacaggaattaaccccttgttaatccataaaacttaaaggagcgggatccaatcttctggcttccctgggccacgctggaagaagaattgtcttgcgccacacataaaatacacgaacactaataatagctgctaagctttaaaaaaattgcaaaaaaggaaaatctcataattttttgtttgttgtgaggtggagcctcactctgtcacccaggccggagtgcagtggcaccatcttggctcactgcaacctctgcctcctgggttcaagccattctcctgcctcagcctcccgagtagctgggatgataggcgtgtgccaccatgcccagctaattttcgtatttttagtagagacggggtttcaccatgttggccaggctggtctcaaactcctgacctcaggtgatccacccacctcggcctcccaaagtgctgggattacaggtgtgagccaccgtgcccggccaatgttttaagaacgtttacgaatttgtattgggccacattcaaagccttcacaggctgcatgcagcctgcaggccgcggttggacaagcttggattagagaaatctacagagacaaactagtgacttagtagccctctgatagctcatgatttgcaagaaacttaggatgactatgtgtaaagaccacaaacatcaatttaactgaatggttcccgccacactggaatgaggaagctgagcaaactcagaggactctaagaaagggctgatgtcatctgaactgttcggaattataaactcctctaaacatgtttcaaagccagaacttgtaggagttgttctgatacacggattaaaagagggatgacaaagtgtctgtcccccacactggtcaaagggacaggtcattgttatgctggcaatgcaggctgctgaaaagaatgtatctgtcaaaagtaatcaaagtaatgaccccagaaggctccagaaacagactggtaaattcaggttgctttcagacttccacaatgctggcacacaaggggaaagacaaaactaacatttacagagcattatatttgatattacatttaatccccattaaaaagatactatttcccgtttcactagtgaaaaagttgatctttcaaaggttaaattatttaacaccaaggtcaaagggtaagttggagagaccagattcaaacccagtctgacattaaaacatgtgttttccccccacatcgtctcctgctaataacctcaaatctaaaaactgacttgccctacaccttgagccccatcctacaaactctccctgacgttattaattcagctgtcactgtgcacctacaacgtgccagacaccatactcctcaacactctgtaggcacagaaggaacagataaaaatccctaccttcatagatattattctaggggtaacacaggtaaataaaacattaaaatagttttcacatagtagcaaattccatatagcaaaataaaacagaagaaggaatagcaaatgagggagatgccctcttaaacatggtgctgagggaaggcctccctgagaaagatatcatttaccccaaaaataaaaaagcaagtaatagaaaaaacaggtaaaaggtgttctagacacttaaacctgccacattgagaactcagggttctgatgcaaaacctcgctgcatagaatgcattaacttatttttatacatttaaacaaacaaactctacttaagaactgtgttctaaaggaaggagcatattacaggaaggcaatttttggtcagagtagacacacttaaaaactaaacctattgaaagaccaagaacaactgaaagtctttgctttgtcagatttttgaccaaaaggaaaattaaagaaacacaccgtgcccatccaatgatttcaccaaggaattttaagagagaaaatcctacttcttcctcacccagtagccagtgaaatgactgagcaaattcacaagttcactggggctgctttcatgtaacacagggacaacacatgacagacacagtggaaccctacaggttgcctagtatttgaaagactgtgaagaggaggagatgtcaaaattcaaagtcttaaatgatgtagttttaagtatgttcagcaatttcaccactcagtagtaaagccagctacagttgaaagcaatcagaaatttgaggggtgtgaaataagcagaagcacagaagttaaggatttgtattcttcccacattttccactttattttatactgctgagaaaaaacaaatttaatagttttctgctgtataagagagacacattcactttatgtcacagtaagagtcactcaattttaatacaactatctcaatgtataaattaacattctcccccctgcccacacatagtaagtctcttatgatgttgctgattagagaagcaaaagttgccgctacaattctcttcctgcattttaatataaacaatcatcagtcttttcttcatagagtgcagtgtgggcactatcatcagaatgtaccagcactgggtgtgcaaagtttacaaagattagcaagagcaaaagtgttgagatttttgaaattcatgctgctgcaaagaagtatgtaaaaactcactcaccatagaggaccacacagaaactcaggcatgaagttatatggctgtgtgagtggtttgggagaaggaacggaaagcacttccaccaacctatatgcctgagcaaattaatgcaaaacctcagaagctacaaaaaagtttatctacctaaattaaaattggtgtccacagcagtagccagcaaaatgcctgcgaagcgcaaagtggtaaatattttagggtctgtaggtcatatggtctctgttaaacaatatgtaaatgaatgggtgtggctgtgttccaataaaacttcatttataaaaagaggcagcatggtacatccagtcagcaagctataatgtaccaacccccggtctaacactaaccaaatacctcttaataagccaaagaaactgtgtcctcttaggccggaagcggtggctcacacctataatcccagcattttgggaggccgaggcggggagatcacctgaggtcaggagtttgagaccatcctggccaacatggtgaaaccctatttctactaaaaatacaaaaattagccaggcgtgctggcgggcgcctgtaatgccaactactggggaggctgaagcacgagaatcgcttgaacccaggaggcagaggttgcagcgagcctagatcacgccattgcactccagcctgggcaacaagagagaaactccgtctcaaaaaaaaaaaaggaaataaaagtatacaaagtgaaaacaaagaaattaaactgcccttatttgccagtgacattactgtctatgcacaaaattccaaaaatctacaaaaaagcttctagtactaaaaatgagtttagcaaggttgtagaatccaaggtcagcatataacataaaatcaccttcctatatactagcaatcaccaactggaaattgagaagtatcattcacaacagtaccacaaacatgaaataaatgtg

SEQ ID NO: 8 - DNA, 8420 bp, Homo sapiens
tcttagtatggtaaaccttttgaagtagattcaaatgagaatgggaagagagaaaagggagagaagcaacataagaaatctcttttaaggaattttatatagagagaaacagaggaatcagttgatagttggaaattattttaaagaaaatgggttattttaaagaaaaaaggtattacaacatgtttgcactattgtgggaataatcaagttgagacagaaaattattttttaaggaagagtctaattgctgaagtgaaagagaatgaatgagaccctgtgcataagtgtgatcagataggagcatgtacagctcaagtaagaacaggaagaaagagacaataaacatgtacagataggatgggctggtcgatgtggtggtgaaaagacatgcgagttattactgattacttctatttccccagtgaaataggaagccaggttcataaaccaaaatgaagaggagcgaggcagtattggaagttcaggaaaagtaataggtgtaaaaatatgtaaagtagaattaccagggagtatgaagatacatttccaattaaggatgaagaatttaaagtgaggccagccaatacccctgctttgcttcagctacatcagctgcataggttcaggcacagaatacatggaacattgtatttaaatagggcctggattttacaaaagtaacacaatgaagaagagagatgcaaggctatttgagggtgtttgtgggagagattgtaaaatattagctaagtaagaaggggactgcaaattttagtggtataaaggaatgaggaaaagtgtaaatacagtggggtcaaagaatgtttggagccaaggcactagaggcaattagctgaaaatgtaggtgattattggtgagtgacatggtttaaatgaaaagtatagaagggtacaattatccatcatgaaaagttctagggtacaactaagatctgagtagctgaagtagaatgaaagtagaatggacctttccatatccagccaggttcagtgacagaaggttaggaaacaaattataaaccacttgagagaacatatcccctaagttgtttttgctatttttctttcagcatatatttgttggaatgccaactatgttcagttcaattaatatgggcttcttaaataagggctccagcactggataatcctgccatttattttgatacattccatcctgctgctcagatctattggcatctacaggatgtcttttgagaagatgggcattcacatccctatgtcctagcaaatttccaactcagaaaaccacattaggcttctctatatatcttccaactatttcaatggaaaatacaattctctgatttcttcctatgatatttatcaaagagaatggtgcctgccagttctagggtgggggaactcaatacaaatcaccaacctttagatgacaccctgtcttcaaagtgctttcaaagtctggcagaaaaaaagtacccagtggctataagaccacccaggagttcagtcatgcattctaagtagcagatcactggaatgtaattggctagtgagttcattttactcttctcttcttggtcacatgttaccgcccttgtaccctgcacgttctctttcccagacttacaaagcatgttctcttgaattcgttctctttttaaattcacacagtcttaatgattcttctttcacaagagtctttcactcttacaattcagttcaagtcatccacatgcttattatgagcaagggtctgggacttaggggaaaagggaataaaaagatgaatgaaatgtgatccctgcagtccaagagcttgctgtgaaaaaggaagtttggcttacattgcctccctaatcccttggctaggccagaacagaatattgtctaaaacctcctcacgtcagcagtcctctggggtggtgactggaagtagaatttaaacaaaaatataattgacacataataattgtgcatacttatagggtacaatctgatgtttcgatatgtgtttaaatgggtgcattgtgtaatgatcaaattgaggtaatttatccaccaccttgaagagagatttttcaatattctcattgcgaagaagcaggaatttttagcagacaactgagatgcttcttgttcacactaagtcattctgacgatggatttacataacttgttgttttttttgtgtgtgtgtttttgagacagagtcttactttgtcgactaggctgaagtgcagtggcacaatctcggctcactgcaacctccacctcccgggttcaaacgattctcctgcctcagcctcctgagtagctgggattacaggtgcatgcaactaggcctggctaatttttatatttttaatacagatgggatttcaccatgttggccctgctggtgtcaaattcgtggcctcaagtgatctaccagctgcggcctcccaaagtgcagggattacaggtgtgagacaccaagcctggtacatttacatttcttatctggatctttcctttagtaagtgctaaggaatcctacttcccccaatattttttcctatttcaatgttttagcatgtatcatgttactactttgcagacatttgattttcccctttgtttactgtaaagtatatttttatagcctttgtaatagaagtattctaaaatctgcctgcaacctatctttctgactctgcattttagggaataattctctgttgtggaatgaaaaaaaaaacagagcctgtggagtcagagatctcatttcaaattatagttatccctaggaataaatctgagtgacaggtagtatagtataataataagtataaagctatggttaaggaaaactcaacaaccttatctgtaaattgggatgacaacagcctacgtcaaaaaaatgtgaaggtaaatgagataatgtaaggctgatacttagtaagcaatttaaaaacacccaaaaaactattgccatgattactctacttactctatttctctatgctccaggcaaatgaactactaatgacccaggggtccttccccattctcttcttcacaaggaaatattctctctctgtgtgctgtttattaaaatctactgccccttttagaagcctttccagatcatcccatggccaagaacgatcgctgcttcctcttctttacatacagatgtttttctcctgcttgacaattatttttgtgcaattattttccttttgattgtgtttttaatgtcccccccaccccacaattttccagactgtttgctccacgagagaggagaccatcatctctgtgctcaccgttgtatgaccagtatcctgaggagtggctgttacataattacatcaggcactcaataaaaatttgatgaataaacactggattttaaggcaggtatcatatcttacatagcatatcatatcttacattttatgtccctcacataaataccacagagtgaagtatatgacagataaggtcatttctcttgataagtacatagtccagtctgaaacagatatgccaaaaaaaaacaaaactggagtaaacaagatgaattgttttaatagaggcactgtattagtttcctaggactgccagaacaaatcacctcaaacttagtggctgaaaacaacaaaaatttattgtctcacagttatagatgttagaagtataaaattaaggtgtcagtgggattggttccttctgggggctgtggaagagaatctgtcccaagccttcacactgtaaagtacagtactggagggataggacttcaacttgctctatctcagatagagaggagccatttgttgtgaattgagaagaggggtatgttgaatccataataagcacataaaaacttggctggttcataggagaagtaacatgtttccagctctagtaaaaaacaaattgaagtggcctataaaaaggtacagagtacgacagaatgaaaaataaatgaacaagaatacagagaggatgtggtaaattatcatgtttccctaatatgttattggacactaaatggtattagaattatttatcaataataattctaaactgttgcaattgaaagaatatattaagtggtgttatatgagaagtgccagggcattctcatttctgtccaatgggagaaacattttcgtttgagacctccgtgaataatacagtcttttagttaggagagctgcattttgagtggtgcaggcagaatggcgatctctcacccacacaaacactaagatagagagagacagagacagagacagagacagcagagagagacagagaaaggaagtacaggtactcagatagagataagccatttcttgacattaagaaataaagtagaatccattggagggaaataaaactgcctcaggaacagagttaattcacatacacatgcaggtaaacacacactgcttgatacttactgtggactttgaaaattatgaatgtgtgtgtgtgtgtgtgtgtacattcagccctccatatccatggattttgcattcacagattcaaccaaccatgaattaaaaacatttggaaataacaaacattaaaatataacaatacaacaataaaaataatacaaataaaaaatatagtgtaacaactgtttacatagcatgtatgttgtattaagtagtataaatctagagattacttaatgtataccagaggatgcataggctatatgcaaatactatgccactttaaactgataagaacagatactaaacttcatcttagccaaaagtcagagaaacaatataactatgccattttacataagggacttgagctgagcatcctcagatttcagtatctttggagttcctggaaacaattccttgttttatatatatatatgtgtgtgtatatatatatatatatatacacacatatatatatatatatatatatgatagctactgagtgacaggtgatattataccataccacttgtcactcagtagctgtatatgcatatgtatatatatacatatacatatatgtgtgtatgtgtatgtgtgtgtgtgtgtgtgtgtgtgtgtatgctgtctttcctcggtatcacagggaattggagatatatatattcttttcagtacaaaaaaaattgaacacagatgggtatggtaccagaacagaaggtaaagacacatgaaaaaaatttgcaacaacatgaatggaactggagatcattatttgaggagaaataatccaggcacagaaaaacaagcattttattattttaggtgaaagacaaacattttattttaggtgaaataatccaggcacagaaagacaaacattgcatgttctcatttatttgtgggatgtaaaaatcaaaacaatagaacgtatggaggtagacagcagaaggatagttaccaaaggctgcaaagggtagtgtaggctttgagggtgaggtggggatggttattgggtacaaaaaatagttagaaagaataaataatatctagtatttaatagcacaacaggttgactatagtcaaaataacataattgtacaatttaaatatgaaattaaatatatatacaagactagaacaccaagttgaatgactccagcttgcgaaacccacattgatcaccatgcttgccccaagggaagctgtacaatgtctggctcgtccagaaccccatcatttatcactagcaatctattgtccataatcatgtttaaattaatagcattttaaaggtacaaatattttttaaaaaacaaataattatttaattcgccttttaaaagctttttaaaaacgtttttaaaaacttttttaaagtcctgaggactattttctttaaagtgctcagttacagagctccatatattgggctatgatagccttacctgattcttgccaagaatctagtgcccagaaaatgcaaatacaaagtaagcaactgaaaaataaacaaataagttggaggtatgctacctgttgaaatatgacctagcgcaaacacctatgccacttgcttatgaaatcatataggttttcggtgtgcagttttgactgaatgagggagtttacgctggaccacaagggggcccctctgtcaataacgtactccatttgtgtattaagtcaaaaatgaaatggaagagaaaagaaacatcgatgaccccaagtctctttaattgaatggaggtaaaagggaaacaacgaatgagaaaagtactctgcccttttaagaatcttgcattcacattcctgatgaagttatttttcctcctctcactgattcccatttcactctattacatagcaccgtgttccccaggagctcctgaatgaaggacatcactcagctgtgttaagtatctggaacaataaatatactagtttcaatgtctaggctatgggtattcctttttactgaaggtatgacatatagctgcccaggcctgactaaattaatagtaataataattaataatggcaaatttttattctattaagttacttggcttgacttgtagaaatagcaacattcatctgaaatgccccctcctacacttatgtctaaggacaaatcccacatacaccacagataacttcattttacatgttttattctgttaccaaactaaatttttatcatatagtctgttgctcactgaactcttcagtaattctcaacataccatgtaaagcattaagcacagttccaacacagagcaaatgagcaataactgttagttattataacattattatgtgttttcagtgcattaaaccactggtctgatacctagcccaacattctattaaaccacataatccagttgaataatatatgataatataataaaatggcgataagtgctaaatatccagatagaaacacagatggaatcagacagctttcccaagaaatagagaaaatagtagataggcgatctaggcctaagcactctaagcagaagctaagttatcacaggatatcttggcaatctgtggcacgtgaacccttttcttctggagtctggaactatgttgcaactctcactttctccctatctagagactcagtttgttcccttgtgattatcagcagttgagaaatccttagaccttctgaaaggactactttttaaatttatatatataatatttaaaatacatatctttatatataatatatatttaaatatataatatttaaattaatatatatttaaatatataatatttaaattaatatatatttaaataaataaatttatatttaaatatataataattaaaatatatttttaatgaacagagagtaaaggattattttgaagagaaactcctggttcccacttaaaatcctttcttgtttccaagtttttcaaatggagccctctttaccagcttgccccctcagagataagctgttcccctacttattcagatctgagatctgaaaacattccttttcctgtgagttcagctaggacaaagatggagctttttgataaaatttggcaaacacattttttaaagatgaaaatttttaaaaattgaaaaaaaaacatttatagaaagagacttctaatccaaatttaacttctcaaactatgttttgaccggctagcataatgtttcagtctttctggagaatgccccttgaaactgttttcttctacacaacttcctcctttcctttgactttcctgctctggaagggaagaacaggaagaggacagatcaaattactcaagaggaaggacaagaaataaggaaccaaattatcaacaattggagaaagaaagctgatgtcagtatcatttcatatatgattatgtcagagtcaggtggataagccaatcctgttgaatagcatacttttcctgctactcctgaagggtaaagaggtctttctcttacaaagccgtcctagctagtaatcttacaggtgcaaaaagcttgttttcatgttatttcttagtaactcaaaatacctctaaagttatacatattatgaaagtactacagtcacagtgctgagaaaaggagtaaataagacaatgtatataaaaacacttggctcagcccctggctctgtggttgataaatattaagttagtattcattattattataatttccaaagagtccattaaaagatatagaagaagggaggcagcaataacactaagagaaaattccattatctccaactatttatcctctagcccaaaataattgccattagaaagagcaactttaacaaaaattttaagttgcaatagatgttcaactttaaatccatcccagaaaaatttctaaccaaaggagcatagaagatttgatcttattttctaagtagtatagacttaattgtgagaacaaaataaaaacttg

SEQ ID NO: 9 - DNA, 8475 bp, Homo sapiens
gcataacttgtaagaaatggagtgaggtctcagttcaaactggcttctgtatgacttcaaagccaaagtcagcaacttagaaggcaaaaattataatttagttggcaaatacgagaaaaggtcagaaacacatgaaatgaagctcaataggaacacttacagggtagcagggtagtagcctagggaaaaaagtcagacactaaaattgtttaaataggtaagttcaagggacaggtaaagaccttagtgggtaagaagccaatcagcagacgaactgcaagcaagcactgtctctctttcccttctgtctcctcttgtagtaactgaccacaattaaggctgcctaggggaataatgaagtaatcctcctattatcagcaatggtctgatccagtgccaggcaccacagacaacttggtgttcagagaagatccttcaagatgaacaaagggtcaaaataaaaaattctagaagagagaagactgatcacaatttaatgtaaggcttggaaggaactgatctctaccttccttaacatctcaagaacttcctcagattcattggatgttgagtgtgtgtgagtctagtagaaaaatgaatttttgtttcttaacttggatatgtgattaggatgttaataattaagtctgggctaatattgaaggtatcttatgatgggcttcttaaagcattgatcacaaagactgcatgttcataaactgagctgcacttgttaggattctagatgtttgaaatttcttgtgttattttggtctcagatttctagacaaattttctcaaattcctatttcactttttgacatatcatgagtgactcaaatgtttgcccttgagtcggaaaacacccagcattaggaataggcacataaacataatacttcaagcttcagatttaagctcaattataaagtgtttaaaggctgtgctgatagttcttctgagtagaattcctacaactatgggtttgtctataataaaatgttcactctatattgaacgccttatttaaaactcgaaatgtgtaagtagtaataaagaaaatatgtcctcctgtaaccaaagctaggaccgattacatgttcacttgactgacagatacaatcacctatattaggagcaatcagcacttccttacaaactaacaacttgagatgtagtgttcccattggctatgaagattttctttatttactcagaatagtctgtaggatctgccagctgcccctgattataccagctgcacccaatgatcacagtgaacattattttacattctaaataactggtgcaaggtgagccatggttttctgagtttcctatcacctttgtgtttcaggtcctcaaatgttaatttgtaaagctgctgtttcaggcaaaactaacaaaattagcatctaatcaataaccatactatgtccacccatatcctataacacagaagtaggggaagagtgagaaaggtggaagtggagaaatagaggcccaaaaagaaagttttatcacaggaatatctagatgtcttctgggattgtctgttaaagagctgtgacactcatataaatgcagaattactctctttcttccttgttggttagaaggccaagggtgccatggtaatactaccaaacatatatcaaagcttggcaggaaaaatggtaccttcagaaattttataatctgatatcaaataggtcaagaaatataataaaactagtttctttggtttccttagaaacctggaaaactttaaattagaaacttagaaagctttaaatcagactttgtagttaaaaaaggaaattttagttccttccagcattagaattccgtgattctctgactctgagcctggattaaatctagcccagctgagtggaaacttaagtaactagctggttgcctttagtgatcttccactttatggctgcttccgcctaagaagttcatcatcgtgacttactttctttggggcaaagtcgtgactaactttctttggggcaaagttggaaagcagaggtcaaagtcaatcagaaatgggacaaactcacttcctactgcctggtgaaggggccattttcagtagccccttttcaagattagtttcattcaagatttgataagctgttttgactttactatagatcttattatccatgtcagttaagtttatgcttccactaaatctatctgaattcaaaaggtaaaaagctaatgctcagtcttatcagatttatcttatttattaatagaatgtggatttttttaagcatataacaataatagtaatgataggaccataaatgtggatggctctttacaagtcactaacattacataaattcctcaacaacacactctgaggccataacaaacttttagaaataacacaattggctacggaactccagccatctagcttcatgggctcccactttaatttcaaaacaacagaactgtgcacattcatttacatgattagggcagagcttaactgtatctcatgtagcacctacatcattcttcagacaaacttattgccttttacagacaagaaaactggggctcaaaaaaggacttgcttataactggctaataaagaggaactctgggttcaaagtgagtccaattctttcttccacccacagcttctgctaaagtcattacagaaatgcatagagcagttcttccacgttattgcttaggtttctaaagagcagtgacctaatacaacatgctctataatttattactgatttaactatttcactaaggattcacttttaacttttaacttgtaaatatgtctaataaacaccactgaaatagcaacctctttcttcatggccttgtggttgtaaagcaagctagtaatatatgtctgtggatttgtgctaataaagttctatacacctcattaattccacaaatcctactgggtatttcttatctgccagatcctacgctaggtactggatacacagtactgaacaaaatgggtacaaatgagcctcacagagcttgtttcattgaaaagcagagagatacacactaatcaacaaattaatagtaacacactacgatgtgttttgaaggaaaattagagcatcaaagagacggtgttagcaggtggaggggagctcttttagatggagaatgagaatgcctccctaaagacatgggaataaattgagatcacaaaaaatgagaaatagccagccttgagaagagcagaaggaagaacattcaaaggaaaagaaagtgcatactggaaagcctgaacactagagtttggtgtatgtaaggagctgagcaatggtcacttgtgtgataagatgtgtggatgtggggtggggggcaggggtgagtcccacgcagctcttaagtgtgtcctcagactcctgtggtttccatcagccacaacctgaataactgtgtggtaatccaaaaatgattacagattaaacatataaaaatatcattacacccatagtacctaagccaaggacacagtattctatcttttcaatgaagatctgcatgaagtaaaattattatatataattttaggtattgatatagatacatcagtggatagatatagatatgtgtctctggtatagaaaaaagttttaaagggatattaaaagttcttatcttgcagggttgaagattgtggcaactttcatttctttttaattttaagaaaaaagtggtattatgggggattagcatgtttgtgggtatatgtatatttttaattaaaaaataaacaacaaaatgaaaacgtttttcttctatgaaagcctaataagaagaaatttcagctgttttaacttagggagctaaaaacatcaaatccaagaatgttctctggaactgagctcaatacatttttatttgagtaagaattggatacatttccatccccttggggctccagtctgtcaatattttacttttcagcgataaaaagacacatgtagataatcacagtgacctcagtaactttccttctcttatttaagtttattttatttctatcgtagttttccctgttaaagattttttctttttgcttacatatataattttagagaataacaatgcacacacaaaaaattcctcttgttctgctagacctggactttttctctaatatatatctccattttttgtcttttttcagacgtattttggaagcaaaggagagaattgctatatagctgacttcctcttctcatcaacagtgttttaacagtttttaagcaaaagtcagctttgtttatctaagattttttttgctggcatttaacctacccctgcctcccctttcccaagtccacttcagccaacctctcattcgacaggtaccaccctctaacataactgaaataatgtctaccattactggatcttgctagcaaagaatctcaaattttcccacttggttgtaaattattttgtaatctctagtgtttaaggtgcgcttgtcctatctaatcccctccctggcaggacaccttacagaacctaccccttacactagtcattaagcaccatcagggacggatggctgtgtcactggtctgtttggtattccctactgatcctaccatgtggtgattatctatgacttccctaatccctggctgccttagctgggactggctgacatgcttctcaggttgccgctggctttacagtcctttactgcccatgccactttggagataggcagggctagtacttttctatataagcccccaaacttgactttgtgtttcacagtaggtgaaaaagttgggtctcttttcttttacttttctttccacaagatgataaagctaggggaagcctgtggacatggtttatttctgcaactgcaatgattgattggtgcttcctgctgcttacttcctaaactttgtgctcagtgtcagatccctagcagtttctatcccctgctctgctaaaaaagaatggatgttgactctcaggccctagttctttttaattaaattgtatttttgttatcattattattattattattttgagatggggtcttactctgtcgcccaggctgaagtgcagtggtgcaatcacagctcactgttttagcctcctgagtagctgggactacaagcgtcatgccaccatgcttctttttaattttttaaaatggttttctgccttcaattctaagcacttctcaattgtaaccaagagataatactttttatgaattcttaaagttatcaacagatactcaaagttttagcaaagtctaaatgatattaagcttgtccttattgcccaagtgacttcaatgactatttgttaattgcaaccaagggtcattttttaaatgaatatatattattattatatatataatattaaggtcctcaaatacctaaaagtttagcaaaatctaaataatattgtgcatattcttttattactgtattagtccgttttcatgttgctgataaagacatacccaagactgggcaatttacaaaagaaagaggttcactggactcacagttccacgtggctggggaggcctcacaatcacggcagcttacgggattgttgagaaatgacacttctcaagctggggctaaactatctctgtggtagttgttctgattcaagtattgaattggttttttttgttttttttgagatggagtttcgttcttgttgcccaggctggagtgcaatggcacgatctcagctcaccgcaacctctgcctcccgggttcaagtgattctcctgcttcagcctcccaagtagctgggactacaggcatgagccaccacacccagctaattttgtatttttagtagagacatggtttctccatgttggtcaggctggtctcaaactcccaacctcaggtgatccacctgccttggcctcctaaagtgctgggattacaggcataagccaccgtgcccggctggagcattggtatataaaagctgcctaggtaactctaacctttggccccatacatctgaaggatacctacaatgcacctgaaaaatgcaactgaaacagtagttccctgggaccacacactcagaaagggggtgtatcaggagatctagggaccaggagggtggaagacctaaggcagcactacagatgatggagaaaaacccactggggaggggcgatcctaaccttgagaatcactgagatcatgcagaagtatttgatcctacagcattaatattgtattgtattgtattagtatatatatatagtgtatatatatagtattagtatatatattgtattgtattagcatatatatactaattgtattgtattgtatttatatatatagtattgtattagtatatatatacagtatatatgtatatatactaatacaatgtactaatacaatacaataccatatatatatacactaacacaatacaattagtatatatatatatatatatactaatacaatacaatactatatatatactaatacaatatatacatatatactcaccaagacatattagtggtctgatgtctggctgccacactcatcttctaccttcagctctgctctaccaaatatcatttgtttctgggatctttgcagtccaaggaacttcatccttgatatcccaccccttactaacttttttttttttttttttttttgagacggagtctcgctgtgtcacccaggctggagtgcagtggtgtgatctcggctcactgcaagctccacctcctgggatcacaccattctcctgcctcagcctcccaagtagctgggactacaggtgcccgccaccacaccaggctaatgttttaccgtgttagcaaggatggtctcgatctcctgacctcatgatccatccgccttggcctcctaaagtgctgggattacaggcataagccaccgcacccggccaccccttactaatttttagtaacgtccaaggattaaaggaaatttgccttacctatttaacaggaatcaacagggttaatctcactccctttctaaaaataatttataaacattgcagacaatctcatctatccctgtctaaactgtgtggaattactgccatttaatgtaatcagtctactcatttagtttgcctaaggaatttttgaaaaaacagttaaatgaatgacttaatggaataaccaggaagttgaagtctccaatagtaagaatgaactcttgctctctggataatcaaatgggtccttcctccttcaggtagatcatgccatttcctcacttacactgaacaggtaaacaacataattactgacttcaacttctagttaattccttcttttatcactgagtatcctttggctgggagttttgttggctatgctgccattttttctagttatcacagtcctataacataccaatccttcaatataactcatctttaaattgtggttttaccttctcaagaagttattaattatgccagtgctaaatcttctaaaatgattgttgacttgttgattagcccccatgcaattcccctctcccgtccctcagcacgtaaggaatggccctttgcttacttccacagatccttaaatctaccagttagaagctaatagcctacctctctaccaggaaggaactgtgggctggaacataatacatgttgacttataatttcttagaaaattgtgtgagaaacatcaaactcctgattccaggatatgccaaagacacatcattaaaaagcaaaacaaaacaaaacaaacctcatttgacgttgctagtagtggcatatttcatcaagatcagctcaaataaatagaagtgagattttcacacaaattagactgtagtgcttttttttttaacttatctttaccatatgatttttaacggtaaaaaaaatcgtttgagatattagatgtataatatttatcatccaattacttcattagttcaatcttttttcaatggcgctcctgcatctgagaataaggtcagaaaatttcatgttctgatttcatgctgattttcagaagaaaaatgttagttttgtatagaataacccatcctaagaaatacatttcttattatatttcttatcttatatttcttaggacaatgagctattcaaagggtgatgataaccagcaccatcagtcagcattatctaagaataagaatctgtgtttctacatacagacctcctaaaaaggaacctacacttaacaggattccccaggcaatttggatgcacattaaagcttgagcaacactgcattagaaagttagttttccatcacaaaaacagtaacaaaaggaatataaagtaagttactttaataatataagaagaggggcaggccgggcgcagtggctcacgcctgtaatcccagcactttgggaggctgaggcgggtggatcacctgaggtc

SEQ ID NO: 10 - DNA, 27 bp, Artificial Sequence
  Synthetic
ttcgcggccgccaaggctgaggcagcg

SEQ ID NO: 11 - DNA, 30 bp, Artificial Sequence
  Synthetic
ttctctagaggttgctgaaagaaaaccaaa

SEQ ID NO: 12 - DNA, 34 bp, Artificial Sequence
  Synthetic
ttcgcggccgcaacaacctaactaatagctgtcc

SEQ ID NO: 13 - DNA, 35 bp, Artificial Sequence
  Synthetic
ttcgcggccgcaaccttcgcggccattttgtcctc

SEQ ID NO: 14 - DNA, 33 bp, Artificial Sequence
  Synthetic
ggtgaagcttatacaaacgttcagaaagtctaa

SEQ ID NO: 15 - DNA, 33 bp, Artificial Sequence
  Synthetic
ggtgccatggggttgctgaaaaaaagaaaaatc

SEQ ID NO: 16 - DNA, 33 bp, Artificial Sequence
  Synthetic
gggtgacgtcatacaaacgttcagaaagtctaa

SEQ ID NO: 17 - DNA, 35 bp, Artificial Sequence
  Synthetic
gggtgcggccgcagaccttccaatttaaacgccac

SEQ ID NO: 18 - DNA, 33 bp, Artificial Sequence
  Synthetic
gaggtctagaggttgctattagaaaaaaaaagg

SEQ ID NO: 19 - DNA, 35 bp, Artificial Sequence
  Synthetic
ggtggcggccgccttttgatagccttcctcacatg

SEQ ID NO: 20 - DNA, 33 bp, Artificial Sequence
  Synthetic
ggtcgctagcggttgctagaaggaaaaaaaaaa

(19) United States
(12) Patent Application Publication
(10) Pub. No.: US 2021/0317498 A1
(43) Pub. Date: Oct. 14, 2021

(54) PROMOTER of Hspa8 GENE

(71) Applicant: Daiichi Sankyo Company, Limited, Chuo-ku,Tokyo (JP)

(72) Inventors: Kenji Masuda, Gyoda-shi (JP); Yuto Nakazawa, Hanyu-shi (JP); Kazuhiko Watanabe, Tatebayashi-shi (JP); Maui Nishio, Kumagaya-shi (JP); Takeshi Okumura, Sano-shi (JP); Koichi Nonaka, Tatebayashi-shi (JP)

(73) Assignee: Daiichi Sankyo Company, Limited, Chuo-ku, Tokyo (JP)

(21) Appl. No.: 17/266,528

(22) PCT Filed: Aug. 8, 2019
(86) PCT No.: PCT/JP2019/031295

§ 371 Date: Feb. 5, 2021

(30) Foreign Application Priority Data
Aug. 9, 2018 (JP) 2018-149854

(51) Int. Cl. C12P 21/00; C12N 5/10; C12N 15/85

(52) U.S. Cl. CPC C12P 21/00; C12N 2510/02; C12N 15/85; C12N 5/10

(57) ABSTRACT

The present invention provides an approach to enhancing the production of a foreign protein serving as a protein-based pharmaceutical product in host cells such as cultured cells derived from a mammal. The present invention provides transfected cells having a novel Hspa8 gene promoter, and a method for secreting and producing a foreign protein at high levels using the transfected cells.

### TECHNICAL FIELD

The present invention relates to transfected mammalian cells with enhanced transcriptional activity in relation to a foreign protein, which is obtained by using a foreign gene expression vector having a Hspa8 gene promoter, and a method for producing the foreign protein using the same.

### BACKGROUND ART

The development of gene recombination techniques has rapidly expanded the market of protein-based pharmaceutical products such as therapeutic proteins and antibody drugs. Among them, antibody drugs do not cause adverse immune responses when administered to the human body and are under active development because of their high specificity.

Examples of hosts to produce the protein-based pharmaceutical products typified by antibody drugs can include microorganisms, yeasts, insects, animal and plant cells, and transgenic animals and plants.

Posttranslational modification such as folding or sugar chain modification is essential for the physiological activity or antigenicity of the protein-based pharmaceutical products. Therefore, microorganisms which cannot perform complicated posttranslational modification, or plants which differ significantly in sugar chain structure from humans are not suitable as hosts. Cultured mammalian cells, such as CHO (Chinese hamster ovary) cells, are currently mainstream due to their having a sugar chain structure similar to that of humans and permitting posttranslational modification, and further in consideration of safety.

Use of cultured mammalian cells as a host presents problems such as low growth rates, low productivity and high cost as compared with microorganisms or the like (Non Patent Literature 1). Furthermore, clinical utilization of protein-based pharmaceutical products requires administering the pharmaceutical products at large doses. Therefore, a lack of sufficient production capacity thereof has been a global issue. In the case of producing a protein-based pharmaceutical product in a cultured mammalian cell expression system, reduction in production cost has been attempted by making improvements to each production step, because the production cost is higher than that of synthetic low-molecular weight pharmaceutical products. However, increasing the amount of protein produced in the cultured mammalian cell expression system is also a promising method for reduction in production cost (Non Patent Literature 2 and 3). Accordingly, in order to increase the productivity of foreign genes in cultured mammalian cells, many approaches such as promoters, enhancers, drug selection markers, gene amplification and culture engineering approaches have been practiced so far through trial and error. In the case of using CHO cells as host cells, a human cytomegalovirus major immediate early promoter (hereinafter, referred to as a CMV promoter) derived from a virus is generally used for the expression of foreign genes, i.e., the production of protein-based pharmaceutical products (Non Patent Literature 4, 5 and 6). It is also known that a polynucleotide upstream of the transcription start site of the gene (promoter region) of elongation factor-1 alpha (EF-1α) (Patent Literature 1 and Non Patent Literature 7) or a human ribosomal protein RPL32 or RPS11 can be used alone or in combination with an additional heterologous promoter in protein expression in CHO cells (Non Patent Literature 8 and Patent Literature 2 and 3). The heat-shock protein A5 (HspaS/GRP78) gene promoter is known to improve the productivity of a foreign protein in mammalian cells (Patent Literature 4).

### CITATION LIST

### Patent Literature

Patent Literature 1: Japanese Patent No. 3051411
Patent Literature 2: WO2006/123097
Patent Literature 3: WO2013/080934
Patent Literature 4: WO2018/066492

### Non Patent Literature

Non Patent Literature 1: Florian M. Wurm., Nat. Biotechnol. 22 (11): 1393-1398, 2004
Non Patent Literature 2: Farid S S., J Chromatogr B Analyt Technol Biomed Life Sci. 848 (1): 8-18, 2007
Non Patent Literature 3: Werner R G. Economic aspects of commercial manufacture of biopharmaceuticals. J Biotechnol. 113 (1-3): 171-182, 2004
Non Patent Literature 4: Durocher Y et al., Curr Opin Biotechnol. 20 (6): 700-707, 2009
Non Patent Literature 5: Boshart M et al., Cell. 41 (2): 521-530, 1985
Non Patent Literature 6: Foecking M K et al., Gene. 45 (1): 101-105, 1986
Non Patent Literature 7: Deer J R. and Allison D S., Biotechnol. Prog. 20: 880-889, 2004
Non Patent Literature 8: Hoeksema F. et al., Biotechnology Research International, Volume 2011, Article ID 492875, 11 pages

### SUMMARY OF INVENTION

### Technical Problem

An object of the present invention is to provide a promoter having high foreign gene expression-enhancing activity in host cells such as cultured mammalian cells, and to provide an approach to enhancing the amount of a foreign protein, which serves as a protein-based pharmaceutical product, produced using the promoter. If a promoter is found which has promoter activity comparable to or higher than that of a human EF-1α promoter in CHO cells or the like, an approach to achieving the stable and high expression of a foreign gene in mammalian cells can be provided. Accordingly, an approach can be provided which contributes to increasing the amount of a protein-based pharmaceutical product produced in a cultured mammalian cell expression system, i.e., reduction in production cost.

### Solution to Problem

The present inventors have conducted intensive studies directed towards achieving the aforementioned object. As a result, the inventors have found that a polynucleotide approximately 2.9 kbp upstream of the start codon of a heat-shock protein A8 (Hspa8) gene has excellent promoter activity and is capable of markedly improving the productivity of a foreign protein to be expressed in cultured mammalian cells, thereby completing the present invention. Specifically, the present invention includes the following aspects of the invention.

(1) A polynucleotide consisting of the nucleotide sequence shown in SEQ ID NO: 1 or a partial sequence of the nucleotide sequence, the polynucleotide being a Chinese hamster derived Hspa8 gene promoter and comprising a polynucleotide consisting of the nucleotide sequence shown in SEQ ID NO: 6.
(2) The polynucleotide according to the above (1), which consists of the nucleotide sequence shown in SEQ ID NO: 1.
(3) The polynucleotide according to the above (1), which consists of the nucleotide sequence shown in SEQ ID NO: 5.
(4) The polynucleotide according to the above (1), which consists of the nucleotide sequence shown in SEQ ID NO: 6.
(5) A polynucleotide consisting of the nucleotide sequence shown in SEQ ID NO: 2 in the sequence listing, the polynucleotide being a human-derived Hspa8 gene promoter.
(6) A polynucleotide consisting of the nucleotide sequence shown in SEQ ID NO: 3 in the sequence listing, the polynucleotide being a mouse-derived Hspa8 gene promoter.
(7) A polynucleotide consisting of the nucleotide sequence shown in SEQ ID NO: 4 in the sequence listing, the polynucleotide being a rat-derived Hspa8 gene promoter.
(8) A polynucleotide consisting of a nucleotide sequence having 95% or higher identity to the nucleotide sequence according to any one of the above (1) to (7), the polynucleotide having promoter activity.
(9) A polynucleotide consisting of a nucleotide sequence having 99% or higher identity to the nucleotide sequence according to any one of the above (1) to (7), the polynucleotide having promoter activity.
(10) A polynucleotide that hybridizes under stringent conditions to a polynucleotide consisting of a nucleotide sequence complementary to the nucleotide sequence according to any one of the above (1) to (9), the polynucleotide having promoter activity.
(11) A foreign gene expression unit comprising the polynucleotide according to any one of the above (1) to (10).
(12) The foreign gene expression unit according to the above (11), wherein the foreign gene is a gene encoding a multimeric protein.
(13) The foreign gene expression unit according to the above (11), wherein the foreign gene is a gene encoding a heteromultimeric protein.
(14) The foreign gene expression unit according to the above (11), wherein the foreign gene is a gene encoding an antibody or an antigen-binding fragment thereof.
(15) A foreign gene expression vector comprising the foreign gene expression unit according to any one of the above (11) to (14).
(16) A foreign gene expression vector comprising the foreign gene expression unit according to any one of the above (11) to (14) and any one or more polynucleotides selected from polynucleotides (a) to (e) of the following group A:

### Group A

(a) a polynucleotide consisting of the nucleotide sequence shown in SEQ ID NO: 7 in the sequence listing,
(b) a polynucleotide consisting of the nucleotide sequence shown in SEQ ID NO: 8 in the sequence listing,
(c) a polynucleotide consisting of the nucleotide sequence shown in SEQ ID NO: 9 in the sequence listing,
(d) a polynucleotide consisting of a nucleotide sequence having 95% or higher identity to the nucleotide sequence of any one of the polynucleotides (a) to (c), the polynucleotide having foreign gene expression-enhancing activity, and
(e) a polynucleotide consisting of a nucleotide sequence having 99% or higher identity to the nucleotide sequence of any one of the polynucleotides (a) to (c), the polynucleotide having foreign gene expression-enhancing activity.
(17) A transfected cell into which the foreign gene expression vector according to the above (15) or (16) has been introduced.
(18) A transfected cell according to the above (17), wherein the cell is a cultured cell derived from a mammal.
(19) The transfected cell according to the above (18), wherein the cultured cell derived from a mammal is a COS-1 cell, a 293 cell, or a CHO cell.
(20) A method for producing a foreign gene-derived protein, comprising culturing the transfected cell according to any one of the above (17) to (19), and obtaining the foreign gene-derived protein from the culture.
(21) Use of the polynucleotide according to any one of the above (1) to (10) for the purpose of expressing a foreign gene in a transfected cell.
(22) Use of the foreign gene expression vector according to the above (15) or (16) for the purpose of expressing a foreign gene in a transfected cell.

### Advantageous Effects of Invention

The transfection of mammalian host cells with a foreign gene expression vector having the Hspa8 gene-derived promoter of the present invention is capable of markedly enhancing the expression of a foreign gene such as one encoding a therapeutic protein or an antibody. Furthermore, the combination of the promoter of the present invention with a DNA element can further enhance the expression of a foreign gene encoding a therapeutic protein, an antibody, or the like.

### BRIEF DESCRIPTION OF DRAWINGS

FIG. 1 schematically shows humanized antibody gene Y expression vectors pDSLH3.1-Hspa8-Y and pDSLH3.1-hEF1α-Y containing a Hspa8 gene or human EF1-α gene-derived promoter as a promoter for antibody H chain and L chain gene expression.

FIG. 2A The amount of antibody produced in fed-batch culture using a humanized antibody Y-expressing stable pool was compared between expression under a Hspa8 gene promoter and expression under a human EF1-α gene promoter. FIG. 2A shows the number of viable cells on each day of sampling.

FIG. 2B The amount of antibody produced in fed-batch culture using a humanized antibody Y-expressing stable pool was compared between expression under a Hspa8 gene promoter and expression under a human EF1-α gene promoter. FIG. 2B shows the amount of the antibody produced on each day of sampling.

FIG. 3A The amount of antibody produced in the fed-batch culture of a humanized antibody Y-expressing stable pool generated using a Hspa8 gene promoter of each promoter length was compared with that for a human EF1-α gene promoter. FIG. 3A shows the number of viable cells on each day of sampling.

FIG. 3B The amount of antibody produced in the fed-batch culture of a humanized antibody Y-expressing stable pool generated using a Hspa8 gene promoter of each promoter length was compared with that for a human EF1-α gene promoter. FIG. 3B shows the amount of antibody produced on each day of sampling.

FIG. 4A The amount of antibody produced in fed-batch culture using a stable pool generated by a humanized antibody gene Y expression vector using the Hspa8 gene as a promoter for antibody H chain and L chain gene expression and either containing or not containing the DNA element A7 was compared in order to compare between the presence and absence of the DNA element A7. FIG. 4A shows the number of viable cells on each day of sampling.

FIG. 4B The amount of antibody produced in fed-batch culture using a stable pool generated by a humanized antibody gene Y expression vector using the Hspa8 gene as a promoter for antibody H chain and L chain gene expression and using a humanized antibody gene Y expression vector either containing or not containing the DNA element A7 was compared in order to compare between the presence and absence of the DNA element A7. FIG. 4B shows the amount of antibody produced on each day of sampling.

FIG. 5A The amount of antibody produced in the fed-batch culture of a humanized antibody Y-expressing stable pool generated using a Hspa8 gene promoter was compared between species from which the Hspa8 gene promoter was derived. Hspa8, hHspa8, mHspa8, and rHspa8 represent results of the Hspa8 derived from Chinese hamster, human, mouse, and rat, respectively. FIG. 5A shows the number of viable cells on each day of sampling.

FIG. 5B The amount of antibody produced in the fed-batch culture of a humanized antibody Y-expressing stable pool generated using a Hspa8 gene promoter was compared between species from which the Hspa8 gene promoter was derived. Hspa8, hHspa8, mHspa8, and rHspa8 represent results of the Hspa8 derived from Chinese hamster, human, mouse, and rat, respectively. FIG. 5B shows the amount of antibody produced on each day of sampling.

FIG. 6 shows the nucleotide sequence of a polynucleotide which is a Chinese hamster-derived Hspa8 gene promoter.

FIG. 7 shows the nucleotide sequence of a polynucleotide which is a human-derived Hspa8 gene promoter.

FIG. 8 shows the nucleotide sequence of a polynucleotide which is a mouse-derived Hspa8 gene promoter.

FIG. 9 shows the nucleotide sequence of a polynucleotide which is a rat-derived Hspa8 gene promoter.

### DESCRIPTION OF EMBODIMENTS

Hereinafter, the present invention will be specifically described.

In the present description, the term “gene” means a moiety that is transcribed into mRNA and translated into a protein, and is used to include, not only DNA but also its mRNA and cDNA, and the RNA thereof.

In the present description, the term “polynucleotide” is used to have the same meaning as that of a nucleic acid, and includes DNA, RNA, a probe, an oligonucleotide, and a primer.

In the present description, the term “polypeptide” is used without being distinguished from the term “protein”.

In the present description, the term “gene expression” means a phenomenon in which a gene is transcribed into mRNA, and/or a phenomenon in which the mRNA is translated into a protein.

In the present description, the term “foreign gene” means a gene that is artificially introduced into host cells.

In the present description, the term “foreign protein” means a protein encoded by the foreign gene.

In the present description, the term “gene expression unit” means a polynucleotide having at least a promoter region, a foreign gene, and a transcriptional terminator region (polyA addition signal) in the reading frame direction of transcription.

In the present description, the term “promoter” means a region to which a transcription factor involved in the start of transcription of DNA into RNA binds. In the present description, the term “promoter region” is also used. Examples of the promoter can include a polynucleotide from a nucleotide approximately 3 kbp upstream of a start codon to a nucleotide immediately upstream of a nucleotide sequence corresponding to the start codon. The promoter may contain 5′UTR and an intron.

In the present description, the term “promoter activity” refers to activity by which transcription starts through the binding of a transcription factor to the promoter to perform the production of a protein encoded by the gene. Promoter activity can be examined by using, as an indicator, the activity of a protein encoded by a reporter gene, such as firefly luciferase.

In the present description, the phrase “to have promoter activity” means that an antibody expression level equivalent to or higher than that of a human EF-1α gene promoter is exhibited, under conditions similar to those for the evaluation of promoter activity with antibody expression level as an indicator in fed-batch culture as described in (Example 2) mentioned later.

In the present description, the term “DNA element” means a polynucleotide having foreign gene expression-enhancing activity when located in proximity to a gene expression unit or in a foreign gene expression vector comprising the gene expression unit.

In the present description, the term “antigen-binding fragment of the antibody” means a partial fragment of the antibody having binding activity to the antigen. Examples thereof include Fab and F(ab′)2, though the antigen-binding fragment is not limited to these molecules as long as it has antigen-binding ability.

In the present description, the term “identity” refers to the relationship between sequences as to two or more nucleotide sequences or amino acid sequences and is determined by the comparison of the sequences, as known in the art. The term “identity” in the art also means, in some cases, the degree to which the sequence of a nucleic acid molecule or a polypeptide matches when comparing between two or more nucleotide sequences or between two or more amino acid sequences. The term “identity” can be evaluated by calculating percentage identity between a smaller sequence of two or more sequences and gap alignment (if present) addressed by a particular mathematical model or computer program (i.e., “algorithm”). Specifically, the identity can be evaluated using software such as ClustalW2 provided by European Molecular Biology Laboratory-European Bioinformatics Institute (EMBL-EBI), though the evaluation method is not limited thereto as long as the method is used by a person skilled in the art.

In the present description, the phrase “to hybridize under stringent conditions” refers to hybridization under conditions in which a so-called specific hybrid is formed whereas a non-specific hybrid is not formed. Examples thereof can include conditions under which a complementary strand of a nucleic acid consisting of a nucleotide sequence having 80% or higher, preferably 90% or higher, more preferably 95% or higher, most preferably 99% or higher identity to a nucleic acid hybridizes whereas a complementary strand of a nucleic acid consisting of a nucleotide sequence having lower identity does not hybridize. More specifically, the phrase is used to mean that hybridization is carried out in the commercially available hybridization solution ExpressHyb Hybridization Solution (manufactured by Clontech) at 68° C., or that hybridization is carried out under conditions using a DNA-immobilized filter in the presence of 0.7 to 1.0 M NaCl at 68° C., and the resultant is then washed at 68° C. with a 0.1× to 2×SSC solution (wherein 1×SSC consists of 150 mM NaCl and 15 mM sodium citrate), or conditions equivalent thereto.

1. Promoter for Use in Enhancement of Foreign Gene Expression

The promoter for use in the enhancement of foreign gene expression according to the present invention (hereinafter, also referred to as the “promoter of the present invention”) is a promoter of a heat-shock protein A8 gene (hereinafter, referred to as “Hspa8”). The promoter is not particularly limited as long as the promoter is a polynucleotide having activity as a Hspa8 promoter. The Hspa8 promoter is preferably a polynucleotide from a nucleotide approximately 2.9 kbp upstream of a start codon to a nucleotide immediately upstream of a nucleotide sequence corresponding to the start codon.

The origin of the Hspa8 promoter is not particularly limited and may be of mammalian origin. Examples thereof can include Chinese hamster, human, mouse, and rat derived Hspa8 promoters. The promoter of the present invention is preferably a rodent-derived Hspa8 promoter, more preferably a Chinese hamster-, mouse-, or rat-derived Hspa8 promoter, still more preferably a mouse- or rat-derived Hspa8 promoter. Examples of a Chinese hamster-derived Hspa8 promoter include the polynucleotide shown in SEQ ID NO: 1 in the sequence listing and FIG. 6. The nucleotide sequence of SEQ ID NO: 1 is a sequence from a nucleotide approximately 2.9 kbp upstream of the start codon of the Chinese hamster-derived Hspa8 gene to the nucleotide immediately upstream of the nucleotide sequence corresponding to the start codon.

The nucleotide sequences of SEQ ID NOs: 2, 3, and 4 are sequences from a nucleotide approximately 2 kbp upstream of the start codon of human-derived Hspa8, mouse-derived Hspa8, and rat-derived Hspa8, respectively, to the nucleotide immediately upstream of the nucleotide sequence corresponding to the start codon. The nucleotide sequences of SEQ ID NOs: 2, 3, and 4 are also shown in FIGS. 7, 8, and 9, respectively.

The Chinese hamster-derived Hspa8 promoter may have a nucleotide sequence consisting of a partial sequence of the sequence shown in SEQ ID NO: 1. Examples thereof include polynucleotides comprising the sequences shown in SEQ ID NOs: 5 and 6, which are sequences from a nucleotide approximately 1.9 and 1.2 kbp, respectively, upstream of the start codon of Hspa8 to the nucleotide immediately upstream of the nucleotide sequence corresponding to the start codon, and further include polynucleotides comprising sequences from a nucleotide approximately 1.1 and 1.0 kbp, respectively, upstream of the start codon of Hspa8 to the nucleotide immediately upstream of the nucleotide sequence corresponding to the start codon. The polynucleotide shown in SEQ ID NO: 6 is preferred.

The promoter of the present invention may be a polynucleotide consisting of a nucleotide sequence having 80% or higher, preferably 90% or higher, more preferably 95% or higher, most preferably 99% or higher identity to the nucleotide sequence shown in any one of SEQ ID NOs: 1 to 6, and having promoter activity.

The promoter of the present invention may be a polynucleotide that hybridizes under stringent conditions to a polynucleotide consisting of a nucleotide sequence complementary to a polynucleotide consisting of any one nucleotide sequence selected from the group consisting of the nucleotide sequences shown in SEQ ID NOs: 1 to 6, and having promoter activity.

The promoter of the present invention may be a mutant polynucleotide consisting of a nucleotide sequence comprising a deletion, substitution, and/or addition of one or more, preferably 1 to 300, more preferably 1 to 30 nucleotides in any one nucleotide sequence selected from the group consisting of the nucleotide sequences shown in SEQ ID NOs: 1 to 6, and having promoter activity.

The introduction of a mutation (deletion, substitution, and/or addition) into the nucleotide sequence can be performed by an approach known in the art such as the Kunkel method or the Gapped-Duplex method, or a method equivalent thereto. For example, a kit for mutation introduction which exploits site-directed mutagenesis (e.g., Mutant-K (manufactured by Takara Bio Inc.) or Mutant-G (manufactured by Takara Bio Inc.), or an LA PCR in vitro Mutagenesis series kit from Takara Bio Inc.) can be utilized. Such a mutant polynucleotide can also be used as the promoter of the present invention.

The foreign gene expression-enhancing activity possessed by the promoter of the present invention can be examined by using, as an indicator, the activity of a protein encoded by a reporter gene, such as firefly luciferase, or the amount of an antibody produced in fed-batch culture. When the amount of the antibody produced in fed-batch culture is equivalent or higher, preferably 1.2 or more times, more preferably 1.5 or more times higher by use of the promoter of the present invention compared with use of a human EF-1a promoter, it can be determined that this promoter has foreign gene expression-enhancing activity. Even in the case of an enhancement of approximately 1.2-fold or more, a reduction of cell culture scale, culture time and the number of purification steps is expected. As a result, an improvement in yield and a reduction in culture cost are attained. The improved yield permits stable supply of a foreign protein as a medicament. Also, the reduced culture cost leads to a reduction in the prime cost of a foreign protein as a medicament.

2. Foreign Gene Expression Unit

The foreign gene expression unit of the present invention (hereinafter, also referred to as the “gene expression unit of the present invention”) has at least the promoter of the present invention described in the preceding section 1., a foreign gene, and a transcriptional terminator region (polyA addition signal) in the reading frame direction of transcription.

The polyA addition sequence can be any sequence having the activity of terminating transcription from the promoter, and may be derived from a gene which is the same as or different from the gene of the promoter.

3. DNA Element for Use in Enhancing Foreign Gene Expression

Combined use of the gene expression unit of the present invention described in the preceding section 2. with a DNA element can further enhance the expression of a foreign gene. The DNA element for combined use can be obtained by interaction with acetylated histone H3 as an indicator. In general, the acetylation of histone (H3, H4) is reportedly involved in the activation of transcription on the basis of two main hypotheses: that conformational change of the nucleosome is involved such that histone tail acetylation neutralizes the charge thereof to loosen the binding between the DNA and the histone (Mellor J. (2006) Dynamic nucleosomes and gene transcription. Trends Genet. 22 (6): 320-329); and that the acetylation is involved in the recruitment of various transcription factors (Nakatani Y. (2001) Histone acetylases—versatile players. Genes Cells. 6 (2): 79-86). Both of the hypotheses strongly suggest that the acetylation of histone is involved in transcriptional activation. Thus, chromatin immunoprecipitation (ChIP) using an anti-acetylated histone H3 antibody is capable of enriching a sample for a DNA element that interacts with acetylated histone H3.

Examples of the DNA element for use in the enhancing of foreign gene expression in combination with the promoter of the present invention can include A2, A7, and A18.

A2 is positioned at a site from 80966429 to 80974878 of human chromosome 15 and is an 8450 bp polynucleotide having an AT content of 62.2%. The nucleotide sequence of A2 is shown in SEQ ID NO: 7 in the sequence listing.

A7 is positioned at a site from 88992123 to 89000542 of human chromosome 11 and is an 8420 bp polynucleotide having an AT content of 64.52%. The nucleotide sequence of A7 is shown in SEQ ID NO: 8 in the sequence listing.

A18 is positioned at a site from 111275976 to 111284450 of human chromosome 4 and is an 8475 bp polynucleotide having an AT content of 62.54%. The nucleotide sequence of A18 is shown in SEQ ID NO: 9 in the sequence listing.

The foreign gene expression-enhancing activity possessed by the DNA element for combined use with the promoter of the present invention can be examined by using, as an indicator, the activity of a protein encoded by a reporter gene, such as SEAP.

For combined use with the promoter of the present invention, any one of the DNA elements described above may be used alone, or two or more copies of one DNA element may be used. Alternatively, two or more DNA elements may be used in combination.

The DNA element used in the present invention may consist of a nucleotide sequence having 80% or higher, preferably 90% or higher, more preferably 95% or higher, most preferably 99% or higher identity to the nucleotide sequence shown in any of SEQ ID NOs: 7 to 9, and having foreign gene expression-enhancing activity. A homology search of the nucleotide sequence can be performed using, for example, a program such as FASTA or BLAST and the DNA Data Bank of JAPAN as the subject of the search.

A person skilled in the art can readily obtain such a homolog gene of the promoter of the present invention with reference to Molecular Cloning (Sambrook, J. et al., Molecular Cloning: a Laboratory Manual 2nd ed., Cold Spring Harbor Laboratory Press, 10 Skyline Drive Plainview, N.Y. (1989)), etc. Likewise, the identity of the nucleotide sequence described above can be determined by FASTA search or BLAST search.

The introduction of a mutation (deletion, substitution, and/or addition) into the polynucleotide can be performed by an approach known in the art such as the Kunkel method or the Gapped-Duplex method, or a method equivalent thereto. For example, a kit for mutation introduction (e.g., Mutant-K (manufactured by Takara Bio Inc.) or Mutant-G (manufactured by Takara Bio Inc.), or an LA PCR in vitro Mutagenesis series kit from Takara Bio Inc.) can be utilized which exploits site-directed mutagenesis. Such a mutant polynucleotide can also be used as the DNA element of the present invention.

4. Obtaining the Polynucleotide

In the present invention, a polynucleotide comprising a foreign gene encoding a foreign protein whose production is to be enhanced as mentioned later can be obtained by a general method given below. The polynucleotide can be isolated, for example, by screening a cDNA library derived from cells or tissues expressing the foreign gene, using a DNA probe synthesized on the basis of the gene fragment. mRNA can be prepared by an approach usually used in the art. For example, the cells or the tissues are treated with a guanidine reagent, a phenol reagent, or the like to obtain total RNA. Then, poly(A)+RNA (mRNA) is obtained therefrom by the affinity column method using an oligo(dT) cellulose column, polyU-Sepharose with Sepharose 2B as a carrier, or the like, or by the batch method. The poly(A)+RNA may be further fractionated by the sucrose density gradient centrifugation method or the like.

Subsequently, single-stranded cDNA is synthesized with the obtained mRNA as a template using an oligo dT primer and reverse transcriptase. Double-stranded cDNA is synthesized from the single-stranded cDNA using DNA synthetase I, DNA ligase and RNase H, etc. The synthesized double-stranded cDNA is converted into a blunt-ended form with T4 DNA synthetase, then subjected to the linkage of an adaptor (e.g., an EcoRI adaptor), phosphorylation, etc., and incorporated into a λ phage such as λgt11 for in vivo packaging to prepare a cDNA library. Alternatively, the cDNA library may be prepared using a plasmid vector instead of the λ phage. Then, a clone having the DNA of interest (a positive clone) can be selected from the cDNA library.

In the case of isolating a polynucleotide comprising the promoter and a terminator region, the DNA element, or a polynucleotide comprising a foreign gene for use in protein production from genomic DNA, the genomic DNA is extracted from a cell line of an organism serving as a source, followed by polynucleotide selection, according to a general approach (Molecular Cloning (1989) and Methods in Enzymology 194 (1991)). The extraction of the genomic DNA can be performed according to, for example, the method of Cryer et al. (Methods in Cell Biology, 12, 39-44 (1975)) and the method of P. Philippsen et al. (Methods Enzymol., 194, 169-182 (1991)).

The obtaining of the polynucleotide of interest comprising the promoter, the DNA element, or a foreign gene can also be performed by, for example, PCR (PCR Technology. Henry A. Erlich, Atockton press (1989)). The amplification of the polynucleotide by PCR employs a 20 to 30 mer synthetic single-stranded DNA as a primer and genomic DNA as a template. The amplified gene is used after its polynucleotide sequence is confirmed. A genomic DNA library such as a bacterial artificial chromosome (BAC) library may be used as a template for PCR.

On the other hand, a polynucleotide comprising a foreign gene having an unknown sequence can be obtained by (a) preparing a gene library according to a common method, (b) selecting the desired polynucleotide from the prepared gene library, and amplifying the polynucleotide. The gene library can be prepared by partially digesting chromosomal DNA obtained by a common method from a cell line of an organism serving as a source, with an appropriate restriction enzyme to prepare fragments, ligating the obtained fragments into an appropriate vector, and introducing the vector into an appropriate host. Alternatively, the gene library may be prepared by extracting mRNA from the cells, synthesizing cDNA therefrom, then ligating the cDNA into an appropriate vector, and introducing the vector into an appropriate host. In this respect, a plasmid known as a well-known vector for gene library preparation can be used as the vector, and a phage vector or a cosmid, etc. can also be widely used. The host to be transfected or transduced can be used according to the type of vector. The polynucleotide comprising a foreign gene is selected from the gene library by colony hybridization, plaque hybridization, or the like using a labeled probe comprising a sequence unique to the foreign gene.

The polynucleotide comprising a foreign gene can also be synthesized entirely chemically. The gene can be synthesized by, for example, a method of preparing a pair of complementary oligonucleotides and annealing these, a method of ligating several annealed DNAs using DNA ligase, or a method of preparing several partially complementary oligonucleotides and filling the gaps therein by PCR.

The polynucleotide sequence can be determined by a usual method, for example, the dideoxy method (Sanger et al., Proc. Natl. Acad. Sci., USA, 74, 5463-5467 (1977)). Alternatively, the polynucleotide sequence may be readily determined using a commercially available sequencing kit or the like.

5. Foreign Gene Expression Vector

A vector comprising the foreign gene expression unit described in the preceding section 2. comprising the promoter described in the preceding section 1. is provided as a foreign gene expression vector of the present invention. The foreign gene expression vector of the present invention may comprise one of the DNA elements described in the preceding section 3., two or more copies of one DNA element, or a combination of two or more DNA elements. When a foreign gene is expressed in host cells using the foreign gene expression vector, the DNA element may be located immediately preceding or immediately following the gene expression unit, or may be located at a position distant from the gene expression unit. Alternatively, one foreign gene expression vector comprising a plurality of DNA elements may be used. The orientation of the DNA element may be in either the forward direction or the reverse direction with respect to the gene expression unit.

Examples of the foreign gene can include, but are not particularly limited to: reporter genes such as the secreted alkaline phosphatase (SEAP) gene, the green fluorescence protein (GFP) gene, and the luciferase gene; various enzyme genes such as the α-amylase gene and the α-galactosidase gene; genes of various interferons such as interferon α and interferon γ, which are pharmaceutically useful physiologically active proteins; genes of various interleukins such as IL1 and IL2; various cytokine genes such as the erythropoietin (EPO) gene and the granulocyte colony-stimulating factor (G-CSF) gene; growth factor genes; and a gene encoding a multimeric protein, for example, a gene encoding a heteromultimer which is an antibody or an antigen-binding fragment thereof. These genes may be obtained by any approach.

The term “antigen-binding fragment of the antibody” means a partial fragment of the antibody having binding activity to the antigen. Examples thereof include Fab, F(ab′)2, Fv, scFv, a diabody, linear antibodies, and multispecific antibodies formed from antibody fragments. Also, Fab′, which is a monovalent fragment of antibody variable regions obtained by the treatment of F(ab′)2 under reductive conditions is included within the antigen-binding fragment of the antibody. However, the antigen-binding fragment is not limited to these molecules as long as it has antigen-binding ability. Furthermore, these antigen-binding fragments also include, not only a fragment obtained by the treatment of the full-length molecule of an antibody protein with an appropriate enzyme but also a protein produced in appropriate host cells using a genetically engineered antibody gene.

The foreign gene expression vector of the present invention can comprise a selection marker for selecting a transfectant. The transfectant can be selected using, for example, a drug resistance marker which confers resistance to a drug such as cerulenin, aureobasidin, zeocin, canavanine, cycloheximide, hygromycin, puromycin, blasticidin, tetracycline, kanamycin, ampicillin, or neomycin. Alternatively, the transfectant may be selected by using, as a marker, a gene that confers, for example, solvent resistance to ethanol or the like, osmotic pressure resistance to glycerol, a salt, or the like, or metal ion resistance to copper or the like.

The foreign gene expression vector of the present invention may be a vector that is not integrated into chromosomal DNA. In general, the foreign gene expression vector is randomly integrated into the chromosome after transfection of host cells. By contrast, use of a constituent derived from a mammalian virus such as simian virus 40 (SV40), papillomavirus (BPV, HPV), or EBV allows the foreign gene expression vector to be used as an episomal vector capable of replicating autonomously in the transfected host cells. For example, a vector having a sequence encoding a SV40-derived replication origin and a trans-acting factor SV40 large T antigen, or a vector having a sequence encoding EBV-derived oriP and EBNA-1 is widely used. The DNA element is capable of exhibiting foreign gene expression-enhancing activity, regardless of the type of vector or the presence or absence of integration into the chromosome.

6. Transfected Cells

The transfected cells of the present invention are transfected cells comprising the foreign gene expression vector of the preceding section 5. introduced thereinto.

The host cells to be transfected are eukaryotic cells, preferably mammalian cells, more preferably human-, mouse-, rat-, hamster-, monkey-, or bovine-derived cells. Examples of the mammalian cells can include, but are not limited to, COS-1 cells, 293 cells, and CHO cells (CHO-K1, CHO—O1, CHO DG44, CHO dhfr-, CHO-S).

In the present invention, the method for introducing the expression vector into host cells can be any method as long as the method allows the introduced gene to be present stably in the host and to be appropriately expressed. Examples thereof can include methods generally used, for example, the calcium phosphate method (Ito et al., (1984) Agric. Biol. Chem., 48, 341), electroporation (Becker, D. M. et al. (1990) Methods. Enzymol., 194, 182-187), the spheroplast method (Creggh et al., Mol. Cell. Biol., 5, 3376 (1985)), the lithium acetate method (Itoh, H. (1983) J. Bacteriol. 153, 163-168), and lipofection.

7. Method for Producing Foreign Protein

The method for producing a foreign protein according to the present invention can be performed by culturing the transfected cells described in the preceding section 6. by a known method, and collecting the foreign protein from the culture, followed by purification. The “culture” means any of a culture supernatant, cultured cells, and a cell homogenate. Not only a monomeric protein but also a multimeric protein may be selected as the foreign protein that can be produced using the transfected cells described in section 6. In the case of producing a heteromultimeric protein constituted by a plurality of different subunits, a plurality of genes encoding these subunits each need to be introduced into the host cells described in section 6.

The method for culturing the transfected cells can be performed according to a usual method for use in the culture of the host cells.

When the transfected cells are mammalian cells, the transfected cells are cultured, for example, at 37° C. under 5% or 8% CO₂ conditions for a culture time on the order of 24 to 1000 hours. The culture can be carried out by, for example, static culture, shake culture, stirring culture, batch culture under aeration, fed-batch culture, perfusion culture or continuous culture.

The expression product of the foreign protein gene from the culture (culture solution) described above can be confirmed by SDS-PAGE, Western blotting, ELISA, or the like.

8. Method for Producing Antibody Protein

Examples of the heteromultimeric protein to be produced using the production method described in the preceding section 7. can include antibody proteins. The antibody protein is a tetramer protein consisting of two molecules of a heavy chain polypeptide and two molecules of a light chain polypeptide. Thus, for obtaining an antibody protein in a form that maintains antigen-binding ability, it is necessary to introduce both heavy chain and light chain genes into the transfected cells described in the preceding section 6. In this case, heavy chain and light chain gene expression units may be present on the same expression vector or may be present on different expression vectors.

Examples of the antibody to be produced according to the present invention can include antibodies prepared by immunizing laboratory animals such as rabbits, mice, and rats with the desired antigen. Further examples of the antibody to be produced according to the present invention can include chimeric antibodies and humanized antibodies originating from the antibodies described above. In addition, a human antibody obtained from a genetically engineered animal or by the phage display method is also an antibody to be produced according to the present invention.

The antibody gene for use in antibody production is not limited to an antibody gene having a particular polynucleotide sequence as long as a combination of a heavy chain polypeptide and a light chain polypeptide obtained by the transcription of the antibody gene and subsequent translation retains the activity of binding to a given antigen protein.

The antibody gene is not necessarily required to encode the full-length molecule of the antibody. A gene encoding an antigen-binding fragment of the antibody can be used. The gene encoding such an antigen-binding fragment can be obtained by genetically engineering a gene encoding the full-length molecule of the antibody protein.

9. Methods for Producing Other Foreign Proteins

Examples of the foreign protein to be produced by the production method of the present invention can include the antibodies mentioned above as well as various human- or non-human animal-derived proteins, antigen-binding fragments thereof, and modified forms of the proteins or the fragments. Examples of such a protein and the like can include, but are not limited to: peptide hormones such as atrial natriuretic peptide (ANP), brain natriuretic peptide (BNP), C-type natriuretic peptide (CNP), vasopressin, somatostatin, growth hormone (GH), insulin, oxytocin, ghrelin, leptin, adiponectin, renin, calcitonin, osteoprotegerin, and insulin-like growth factor (IGF); cytokines such as interleukin, chemokine, interferon, tumor necrosis factor (TNFα/β as well as TNF superfamily, etc.), nerve growth factor (NGF), cell growth factor (EGF, FGF, PDGF, HGF, TGF, etc.), hematopoietic factor (CSF, G-CSF, erythropoietin, etc.), and adiponectin; receptors such as TNF receptor; enzymes such as lysozyme, protease, proteinase, and peptidase; functional fragments thereof (fragments partially or wholly retaining the biological activity of the original protein); and fusion proteins comprising these proteins.

### EXAMPLES

Hereinafter, the present invention will be specifically described with reference to Examples. However, these Examples do not limit the technical scope of the present invention by any means. Plasmids, restriction enzymes, DNA-modifying enzymes, etc. used in the Examples of the present invention are commercially available and can be used according to common methods. Operations used in DNA cloning, polynucleotide sequencing, transfection of host cells, culture of transfected cells, collection of a protein from the resulting culture, purification, etc. are also well known to a person skilled in the art or can be derived from the literature.

### (Example 1) Cloning of Hspa8 Gene Promoter Region

The Hspa8 promoter region used was a sequence from a nucleotide approximately 2.9 kbp upstream of the start codon of Hspa8 to the nucleotide immediately upstream of the nucleotide sequence corresponding to the start codon with reference to the sequence of mRNA registered under NM_001246729.1 and the scaffold sequence of the Chinese hamster genome registered under NW_003616190.1 in GenBank.

The Hspa8 promoter region was amplified by PCR with the genomic DNA of CHO cells as a template using the primer set given below and PrimeSTAR Max DNA Polymerase (Takara Bio Inc.), and the PCR product was purified using a QIAquick PCR Purification kit (Qiagen). The nucleotide sequence of the cloned Chinese hamster Hspa8 promoter region is shown in SEQ ID NO: 1 in the sequence listing. Primer set for Hspa8 promoter:

Hspa8-NotI-F:
(SEQ ID No. 10)
TTCGCGGCCGCCAAGGCTGAGGCAGCG
Hspa8-XbaI-R:
(SEQ ID No. 11)
TTCTCTAGAGGTTGCTGAAAGAAAACCAAA

### (Example 2) Evaluation of Hspa8 Promoter by Fed-Batch Culture with Antibody Expression Level as Indicator

2-1) Construction of Antibody Expression Vector

A humanized antibody gene Y expression vector pDSLH3.1-Hspa8-Y having pDSLH4.1 (see Okumura T et al., J Biosci Bioeng., 120 (3): 340-346, 2015) as a vector backbone was constructed. This vector contained the Hspa8 promoter for the expression of antibody H chain and L chain genes, and no DNA element. First, the DNA fragment amplified and purified in Example 1 was digested with NotI-XbaI and then inserted into the NotI-NheI sites of H chain gene expression vector pDSH1.1-hEF1α-Y and L chain gene expression vector pDSL2.1-hEF1α-Y described in Patent Literature 4 to construct pDSH1.1-Hspa8-Y and pDSL2.1-Hspa8-Y, respectively. Next, a DNA fragment obtained by the digestion of pDSL2.1-Hspa8-Y with AatII-MluI was inserted into the AatII-MluI site of pDSH1.1-Hspa8-Y to construct pDSLH3.1-Hspa8-Y. The vectors are schematically shown in FIG. 1.

2-2) Generation of Humanized Antibody Y-Expressing Stable Pool

CHO-K1 cells (ATCC) were adapted to suspension culture in a serum-free suspension culture condition, to obtain CHO—O1 cells as host cells. The CHO—O1 cells were transfected with the antibody expression vector pDSLH3.1-Hspa8-Y constructed in (2-1) and pDSLH3.1-hEF1α-Y described in Patent Literature 4 using a transfection apparatus Neon Transfection System (Invitrogen), and cultured in 5% CO₂ at 37° C. in a T25 flask. One day after the transfection, Geneticin (Life Technologies Corporation) was added thereto at a final concentration of 800 μg/mL, followed by drug selection culture for 1 week. Then, the cells were cultured in 5% CO₂ at 37° C. in a 125 mL Erlenmeyer flask to obtain a humanized antibody Y-expressing stable pool.

2-3) Evaluation of Amount of Antibody Produced by Fed-Batch Culture of Humanized Antibody Y-Expressing Stable Pool

Fed-batch culture was performed in a 125 mL Erlenmeyer flask using each humanized antibody Y-expressing stable pool generated in (2-2). The basal medium used was G13 (custom medium manufactured by IS Japan Co., Ltd.), and the feed medium used was F13 (custom medium manufactured by IS Japan Co., Ltd.).

The change in the number of viable cells and the change in the amount of antibody produced are shown in FIGS. 2A and 2B, respectively. The amount of antibody produced with the Hspa8 promoter on day 14 of culture reached 3.9 times the value of that with the human EF1-α promoter, and thus greatly exceeded the amount of antibody produced with a promoter that is currently frequently used.

### (Example 3) Study on Hspa8 Promoter Length with Antibody Expression Level as an Indicator in Fed-Batch Culture

3-1) Construction of Antibody Expression Vector

pDSLH3.1-Hspa8-1.9-Y and pDSLH3.1-Hspa8-1.2-Y were constructed by substituting the promoter for antibody H chain and L chain genes in the humanized antibody gene Y expression vector pDSLH3.1-hEF1α-Y with a partial sequence of the Hspa8 promoter. In these expression vectors, the partial sequence of the Hspa8 promoter used was a sequence from a nucleotide approximately 1.9 and 1.2 kbp, respectively, upstream of the start codon of Hspa8 to the nucleotide immediately upstream of the nucleotide sequence corresponding to the start codon.

pDSLH3.1-Hspa8-1.9-Y was constructed by the following method: first, the partial sequence of the Chinese hamster Hspa8 promoter was amplified by PCR with pDSH1.1-Hspa8-Y as a template using the primer set given below and PrimeSTAR Max DNA Polymerase, and the PCR product was purified using a QIAquick PCR Purification kit. The purified DNA fragment was digested with NotI-XbaI and then inserted into the NotI-NheI sites of H chain gene expression vector pDSH1.1-hEF1α-Y and L chain gene expression vector pDSL2.1-hEF1α-Y to construct pDSH1.1-Hspa8-1.9-Y and pDSL2.1-Hspa8-1.9-Y, respectively. Next, a DNA fragment obtained by the digestion of pDSL2.1-Hspa8-1.9-Y with AatII-MluI was inserted into the AatII-MluI site of pDSH1.1-Hspa8-1.9-Y to construct pDSLH3.1-Hspa8-1.9-Y. pDSLH3.1-Hspa8-1.2-Y was constructed in the same way as above. Primer set for Hspa8 promoter 1.9 kbp:

Hspa8-NotI-1900F:
(SEQ ID No. 12)
TTCGCGGCCGCAACAACCTAACTAATAGCTGTCC
Hspa8-XbaI-R:
(SEQ ID No. 11)
TTCTCTAGAGGTTGCTGAAAGAAAACCAAA
Primer set for Hspa8 promoter 1.2 kbp:
Hspa8-NotI-1200F:
(SEQ ID No. 13)
TTCGCGGCCGCAACCTTCGCGGCCATTTTGTCCTC
Hspa8-XbaI-R:
(SEQ ID No. 11)
TTCTCTAGAGGTTGCTGAAAGAAAACCAAA

3-2) Generation of Humanized Antibody Y-Expressing Stable Pool

The CHO—O1 cells were transfected with the antibody expression vector pDSLH3.1-hEF1α-Y, pDSLH3.1-Hspa8-Y, pDSLH3.1-Hspa8-1.9-Y, or pDSLH3.1-Hspa8-1.2-Y constructed in (2-1) or (3-1), according to the method described in (2-2). Drug selection culture was performed to generate a humanized antibody Y-expressing stable pool.

3-3) Evaluation of Amount of Antibody Produced by Fed-Batch Culture of Humanized Antibody Y-Expressing Stable Pool

Fed-batch culture was performed in a 125 mL Erlenmeyer flask using each humanized antibody Y-expressing stable pool generated in (3-2). The basal medium used was G13, and the feed medium used was F13.

The change in the number of viable cells and the change in the amount of antibody produced are shown in FIGS. 3A and 3B, respectively. The Hspa8 promoter having a decreased length increased the amount of antibody produced. The value exhibited by the 1.2 kbp Hspa8 promoter was 1.3 times the value of the 2.9 kbp one and reached 5.2 times the value of the human EF1-α promoter that was used as a control. The Hspa8 promoter having the optimized length was able to exert its strong promoter activity and consequently surpassed the human EF1-α promoter in terms of the amount of antibody produced.

(Example 4) Study on Effect Brought about by Combination of Hspa8 Promoter and A7 with Antibody Expression Level as an Indicator in Fed-Batch Culture

4-1) Construction of Antibody Expression Vector

DNA element A7 described in Patent Literature 3 was inserted upstream of the expression cassette of the antibody expression vector pDSLH3.1-Hspa8-1.9-Y constructed in (3-1) to construct pDSLHA4.1-Hspa8-1.9-Y.

4-2) Generation of Humanized Antibody Y-Expressing Stable Pool

The CHO—O1 cells were transfected with the DNA element A7-free antibody expression vector pDSLH3.1-Hspa8-1.9-Y constructed in (3-1), or the DNA element A7-containing antibody expression vector pDSLHA4.1-Hspa8-1.9-Y constructed in (4-1), according to the method described in (2-2). Drug selection culture was performed to generate a humanized antibody Y-expressing stable pool.

4-3) Evaluation of Amount of Antibody Produced by Fed-Batch Culture of Humanized Antibody Y-Expressing Stable Pool

Fed-batch culture was performed in a 125 mL Erlenmeyer flask using each humanized antibody Y-expressing stable pool generated in (4-2). The basal medium used was G13, and the feed medium used was F13.

The change in the number of viable cells and the change in the amount of antibody produced are shown in FIGS. 4A and 4B, respectively. On day 14 of culture, the amount of antibody produced from the A7-containing antibody expression vector was 3.4 times the value of that from the A7-free antibody expression vector. These results demonstrated that the combined use of the Hspa8 promoter with DNA element A7 can effectively achieve high production by synergistic effects.

### (Example 5) Evaluation of Human, Mouse, and Rat Hspa8 Promoters by Fed-Batch Culture with Antibody Expression Level as an Indicator

5-1) Construction of Antibody Expression Vector

pDSLH3.1-hHspa8-Y, pDSLH3.1-mHspa8-Y, and pDSLH3.1-rHspa8-Y were constructed by substituting the promoter for antibody H chain and L chain genes in the humanized antibody gene Y expression vector pDSLH3.1-hEF1α-Y with human, mouse, and rat Hspa8 promoters, respectively. In each expression vector, the Hspa8 promoter used was a sequence from a nucleotide approximately 2.0 kbp upstream of the start codon of Hspa8 to the nucleotide immediately upstream of the nucleotide sequence corresponding to the start codon. The nucleotide sequences of the cloned human, mouse, and rat Hspa8 promoters are shown in SEQ ID NOs: 2, 3, and 4, respectively, in the sequence listing.

pDSLH3.1-hHspa8-Y was constructed by the following method: first, a human Hspa8 promoter was amplified by PCR with human genomic DNA as a template using the primer set given below and PrimeSTAR Max DNA Polymerase, and the PCR product was purified using a QIAquick PCR Purification kit. The purified DNA fragment was digested with HindIII-EcoT14I or AatII-EcoT14I and then inserted into the HindIII-NheI site of the H chain gene expression vector pDSH1.1-hEF1α-Y and the AatII-NheI site of the L chain gene expression vector pDSL2.1-hEF1α-Y to construct pDSH1.1-hHspa8-Y and pDSL2.1-hHspa8-Y, respectively. Next, a DNA fragment obtained by the digestion of pDSL2.1-hHspa8-Y with AatII-HindIII was inserted into the AatII-HindIII site of pDSH1.1-hHspa8-Y to construct pDSLH3.1-hHspa8-Y. pDSLH3.1-mHspa8-Y and pDSLH3.1-rHspa8-Y were constructed in the same way as above. Primer set for human Hspa8 promoter for H chain gene expression vector insertion:

Hspa8-human-HindIII-F:
(SEQ ID No. 14)
GGTGAAGCTTATACAAACGTTCAGAAAGTCTAA
Hspa8-human-EcoT14I-R:
(SEQ ID No. 15)
GGTGCCATGGGGTTGCTGAAAAAAAGAAAAATC
Primer set for human Hspa8 promoter for L chain
gene expression vector insertion:
Hspa8-human-AatII-F:
(SEQ ID No. 16)
GGGTGACGTCATACAAACGTTCAGAAAGTCTAA
Hspa8-human-EcoT14I-R:
(SEQ ID No. 15)
GGTGCCATGGGGTTGCTGAAAAAAAGAAAAATC
Primer set for mouse Hspa8 promoter:
Hspa8-mouse-NotI-F:
(SEQ ID No. 17)
GGGTGCGGCCGCAGACCTTCCAATTTAAACGCCAC
Hspa8-mouse-XbaI-R:
(SEQ ID No. 18)
GAGGTCTAGAGGTTGCTATTAGAAAAAAAAAGG
Primer set for rat Hspa8 promoter:
Hspa8-rat-NotI-F:
(SEQ ID No. 19)
GGTGGCGGCCGCCTTTTGATAGCCTTCCTCACATG
Hspa8-rat-NheI-R:
(SEQ ID No. 20)
GGTCGCTAGCGGTTGCTAGAAGGAAAAAAAAAA

5-2) Generation of Humanized Antibody Y-Expressing Stable Pool

The CHO—O1 cells were transfected with the antibody expression vector pDSLH3.1-hEF1α-Y, pDSLH3.1-Hspa8-1.9-Y, pDSLH3.1-hHspa8-Y, pDSLH3.1-mHspa8-Y, or pDSLH3.1-rHspa8-Y constructed in (2-1), (3-1) or (5-1), according to the method described in (2-2). Drug selection culture was performed to generate a humanized antibody Y-expressing stable pool.

5-3) Evaluation of Amount of Antibody Produced by Fed-Batch Culture of Humanized Antibody Y-Expressing Stable Pool

Fed-batch culture was performed in a 125 mL Erlenmeyer flask using each humanized antibody Y-expressing stable pool generated in (5-2). The basal medium used was G13, and the feed medium used was F13.

The change in the number of viable cells and the change in the amount of antibody produced are shown in FIGS. 5A and 5B respectively. The amounts of antibody produced with the rat, mouse, Chinese hamster, and human Hspa8 promoters on day 14 of culture reached 7.0, 6.8, 4.5, and 2.1 times the value of that with the human EF1-α promoter, respectively, and thus greatly exceeded the amount of the antibody produced with the human EF1-α promoter, regardless of organism species. The amounts of antibody produced with the rat and mouse Hspa8 promoters were further increased compared to the value for the Chinese hamster Hspa8 promoter. These results demonstrated that the Hspa8 promoter of an appropriately selected organism species can exert its strong promoter activity.

### INDUSTRIAL APPLICABILITY

The transfection of mammalian host cells with a foreign gene expression unit containing the promoter of the present invention or the foreign gene expression vector of the present invention is capable of increasing the productivity of a foreign gene such as a therapeutic protein or an antibody.

### Sequence Listing Free Text

SEQ ID NO: 1—Chinese hamster-derived Hspa8 promoter
SEQ ID NO: 2—Human-derived Hspa8 promoter
SEQ ID NO: 3—Mouse-derived Hspa8 promoter
SEQ ID NO: 4—Rat-derived Hspa8 promoter
SEQ ID NO: 5—Nucleotide sequence of Chinese hamster-derived Hspa8 promoter from a nucleotide approximately 1.9 kbp upstream of the start codon of Chinese hamster Hspa8 to the nucleotide immediately upstream of the nucleotide sequence corresponding to the start codon
SEQ ID NO: 6—Nucleotide sequence of Chinese hamster-derived Hspa8 promoter from a nucleotide approximately 1.2 kbp upstream of the start codon of Chinese hamster Hspa8 to the nucleotide immediately upstream of the nucleotide sequence corresponding to the start codon
SEQ ID NO: 7—Nucleotide sequence of DNA element A2
SEQ ID NO: 8—Nucleotide sequence of DNA element A7
SEQ ID NO: 9—Nucleotide sequence of DNA element A18
SEQ ID NO: 10—Primer Hspa8-NotI-F for Hspa8 promoter
SEQ ID NO: 11—Primer Hspa8-XbaI-R for Hspa8 promoter
SEQ ID NO: 12—Primer Hspa8-NotI-1900F for Hspa8 promoter 1.9 kbp
SEQ ID NO: 13—Primer Hspa8-NotI-1200F for Hspa8 promoter 1.2 kbp
SEQ ID NO: 14—Primer Hspa8-human-HindIII-F for human Hspa8 promoter
SEQ ID NO: 15—Primer Hspa8-human-EcoT14I-R for human Hspa8 promoter
SEQ ID NO: 16—Primer Hspa8-human-AatII-F for human Hspa8 promoter
SEQ ID NO: 17—Primer Hspa8-mouse-NotI-F for mouse Hspa8 promoter
SEQ ID NO: 18—Primer Hspa8-mouse-XbaI-R for mouse Hspa8 promoter
SEQ ID NO: 19—Primer Hspa8-rat-NotI-F for rat Hspa8 promoter
SEQ ID NO: 20—Primer Hspa8-rat-NheI-R for rat Hspa8 promoter

1. A polynucleotide consisting of the nucleotide sequence shown in SEQ ID NO: 1 or a partial sequence of the nucleotide sequence, the polynucleotide being a Chinese hamster-derived Hspa8 gene promoter and comprising a polynucleotide consisting of the nucleotide sequence shown in SEQ ID NO: 6.

2. The polynucleotide according to claim 1, which consists of the nucleotide sequence shown in SEQ ID NO: 1.

3. The polynucleotide according to claim 1, which consists of the nucleotide sequence shown in SEQ ID NO: 5.

4. The polynucleotide according to claim 1, which consists of the nucleotide sequence shown in SEQ ID NO: 6.

5. A polynucleotide consisting of the nucleotide sequence shown in SEQ ID NO: 2, the polynucleotide being a human-derived Hspa8 gene promoter.

6. A polynucleotide consisting of the nucleotide sequence shown in SEQ ID NO: 3, the polynucleotide being a mouse-derived Hspa8 gene promoter.

7. A polynucleotide consisting of the nucleotide sequence shown in SEQ ID NO: 4, the polynucleotide being a rat-derived Hspa8 gene promoter.

8. A polynucleotide consisting of a nucleotide sequence having 95% or higher identity to the nucleotide sequence according to claim 1, the polynucleotide having promoter activity.

9. A polynucleotide consisting of a nucleotide sequence having 99% or higher identity to the nucleotide sequence according to claim 1, the polynucleotide having promoter activity.

10. A polynucleotide that hybridizes under stringent conditions to a polynucleotide consisting of a nucleotide sequence complementary to the nucleotide sequence according to claim 1, the polynucleotide having promoter activity.

11. A foreign gene expression unit comprising the polynucleotide according to claim 1.

12. The foreign gene expression unit according to claim 11, wherein the foreign gene is a gene encoding a multimeric protein.

13. The foreign gene expression unit according to claim 11, wherein the foreign gene is a gene encoding a heteromultimeric protein.

14. The foreign gene expression unit according to claim 11, wherein the foreign gene is a gene encoding an antibody or an antigen-binding fragment thereof.

15. A foreign gene expression vector comprising the foreign gene expression unit according to claim 11.

16. A foreign gene expression vector comprising the foreign gene expression unit according to claim 11 and any one or more polynucleotides selected from polynucleotides (a) to (e) of the following group A:
group A
(a) a polynucleotide consisting of the nucleotide sequence shown in SEQ ID NO: 7,
(b) a polynucleotide consisting of the nucleotide sequence shown in SEQ ID NO: 8,
(c) a polynucleotide consisting of the nucleotide sequence shown in SEQ ID NO: 9,
(d) a polynucleotide consisting of a nucleotide sequence having 95% or higher identity to the nucleotide sequence of any one of the polynucleotides (a) to (c), the polynucleotide having foreign gene expression-enhancing activity, and
(e) a polynucleotide consisting of a nucleotide sequence having 99% or higher identity to the nucleotide sequence of any one of the polynucleotides (a) to (c), the polynucleotide having foreign gene expression-enhancing activity.

17. A transfected cell into which the foreign gene expression vector according to claim 15 has been introduced.

18. The transfected cell according to claim 17, wherein the cell is a cultured cell derived from a mammal.

19. The transfected cell according to claim 18, wherein the cultured cell derived from a mammal is a COS-1 cell, a 293 cell, or a CHO cell.

20. A method for producing a foreign gene-derived protein, comprising culturing the transfected cell according to claim 17, and obtaining the foreign gene-derived protein from the culture.

21. A method for expressing a foreign gene in a transfected cell comprising introducing a polynucleotide according to claim 1 into a cell.

22. A method for expressing a foreign gene in a transfected cell comprising introducing a foreign gene expression vector according to claim 15 into a cell.
